(19) United States
(12) Patent Application Publication
(10) Pub. No.: US 2020/0384007 A1
(43) Pub. Date: Dec. 10, 2020

(54) LIPID NANOPARTICLES CONTAINING PHARMACEUTICAL AND/OR NUTRACEUTICAL AGENTS AND METHODS THEREOF

(71) Applicant: Board of Regents, The University of Texas System, Austin, TX (US)

(72) Inventors: Zhengrong Cui, Austin, TX (US); Solange Valdes, Austin, TX (US)

(21) Appl. No.: 16/895,201

(22) Filed: Jun. 8, 2020

Related U.S. Application Data
(60) Provisional application No. 62/858,114, filed on Jun. 6, 2019.

(51) Int. Cl. A61K 31/7068; A61K 47/34; A61K 47/14; A61K 47/26; A61K 9/16; A61K 9/00

(52) U.S. Cl. CPC A61K 31/7068; A61K 47/34; A61K 9/0053; A61K 47/26; A61K 9/1641; A61K 47/14

(57) ABSTRACT

Disclosed are nanoparticles comprising an active compound comprising a nucleobase analogue moiety covalently linked to an omega-3 polyunsaturated fatty acid moiety, or a pharmaceutically acceptable salt or prodrug thereof; a pegylated vitamin E compound; and at least one oil phase component. Also disclosed are methods for treating a subject with a disease comprising administering to the subject a therapeutically effective amount of the disclosed nanoparticles. The compositions and methods are useful for treating diseases such as tumors by delivering an array of active compounds including, for instance, 4-(N)-docosahexaenoyl 2′, 2′-difluorodeoxycytidine (DHA-dFdC) or other hydrophobic and/or lipophilic anti-cancer agents. The compositions and methods are further useful for delivering pharmaceutical and/or nutraceutical agents via numerous routes of administration. Methods of making the nanoparticles are also disclosed.

### CROSS REFERENCE TO RELATED APPLICATIONS

This application claims the benefit of priority to U.S. Provisional Application 62/858,114, filed Jun. 6, 2019, which is incorporated by reference herein in its entirety.

### STATEMENT REGARDING FEDERALLY SPONSORED RESEARCH

This invention was made with government support under Grant No. CA179362 awarded by the National Institutes of Health. The Government has certain rights in the invention.

### FIELD

The disclosure generally relates to nanoparticles, more specifically lipid-based and/or solid-lipid nanoparticles, particularly nanoparticles which can incorporate and deliver pharmaceutical and/or nutraceutical agents. In some embodiments, the nanoparticles are well-suited for incorporation and delivery of omega-3 fatty acids, nucleoside analogues, and/or derivatives thereof, which in some instances can be used as anti-cancer therapeutics. The nanoparticles are generally capable of controlled and sustained release of such beneficial agents, delivery of agents to desirable tissues such as tumors, and can generally increase the aqueous solubility and bioavailability of agents, thereby stabilizing and increasing the effective amount of an agent used in an administered formulation.

### BACKGROUND

Delivery of drugs to desired tissues in vivo presents numerous challenges. One must overcome, for instance, issues related to solubility, toxicity, tissue targeting, stability, clearance, dosing, and many other complications. Delivery of hydrophobic and/or lipophilic compounds present unique challenges because such compounds are not readily soluble or stable in bodily fluids. As such, there is a need in the art for delivery vehicles which can deliver a wide array of compounds, including hydrophobic and/or lipophilic compounds.

Gemcitabine (2′, 2-difluorodeoxycytidine, dFdC) is a nucleoside analogue approved for treatment of pancreatic, lung, breast, and ovarian cancer by slow intravenous infusion (Carmichael, et al., British J. Cancer, 1996, 73, (1), 101-105; Hoang, et al., Lung Cancer 2003, 42, (1), 97-102; Albain, et al., J. Clin. Oncol., 2008, 26, (24), 3950-3957; Ozols, et al., Seminars Oncology, 2005; Elsevier: pp 4-8). To improve efficacy of gemcitabine, a new compound, DHA-dFdC, was synthesized by conjugating docosahexaenoic acid (DHA), an omega-3 polyunsaturated fatty acid (PUFA), to dFdC on the 4-N position (Naguib, et al., Neoplasia, 2016, 18, (1), 33-48). DHA-dFdC showed potent and broad spectrum antitumor activity against NCI-60 DTP human tumor cell lines and was significantly more effective than the molar equivalent dose of gemcitabine in controlling pancreatic tumor growth in several mouse models of pancreatic cancer, including a genetically engineered mouse model that spontanouesly develop pancreatic tumors resembling human pancreatic ductal adenocarcinoma (PDA) and athymic mice with orthotopically implanted human pancreatic tumor cells that are resistant to gemcitabine. Id. The repeat dose-maximum tolerated dose of DHA-dFdC in an aqueous solution was 50 mg/kg in DBA/2 mice (Valdes, et al., Pharm. Res., 2017, 34, (6), 1224-1232). However, DHA-dFdC is poorly soluble in water (intrinsic solubility, ˜25 μg/mL). DHA-dFdC has been formulated into a Tween 80-ethanol in water solution, but the formulation lacked chemical stability (Naguib, et al., Neoplasia, 2016, 18, (1), 33-48).

Drug administration can be performed by many routes, some more desirable than others. It is advantageous if a drug can be formulated for multiple routes of administration, particularly including oral administration. The oral route is often preferred for drug administration due to advantages such as painlessness, easiness for self-administration, flexibility in dosage regimen, convenience, and high patient compliance (Thanki, et al., J. Controlled Release 2013, 170, (1), 15-40). Further, oral product manufacturing does not require sterile conditions that are necessary for products intended for parenteral administration (Date, et al., J. Controlled Release, 2016, 240, 504-526).

In cancer chemotherapy, cancer patients reportedly prefer oral administration to intravenous infusion, especially when chemotherapy is a palliative treatment (Thanki, et al., J. Controlled Release 2013, 170, (1), 15-40; Liu, et al., J. Clin. Oncol., 1997, 15, (1), 110-115; Eek, et al., Patient Prefererence Adherence, 2016, 10, 1609). However, oral administration of cancer chemotherapeutic agents is challenging, in part because the gastrointestinal (GI) tract presents various physiological, enzymatic and chemical barriers, hindering efficient oral absorption (Thanki, et al., J. Controlled Release 2013, 170, (1), 15-40; Lin, et al., J. Food Drug Analysis, 2017, 25, (2), 219-234). In addition, factors such as low solubility, poor intestinal permeability, and high levels of P-glycoprotein (P-gp) in the GI tract wall also limit the oral bioavailability of many cancer chemotherapeutic agents such as paclitaxel, docetaxel, doxorubicin, tamoxifen, etc. (Thanki, et al., J. Controlled Release 2013, 170, (1), 15-40).

### SUMMARY

The present disclosure solves problems in the art regarding delivery of active compounds in vivo by providing for nanoparticles, and methods of using nanoparticles, which effectively deliver one or more active compounds to target tissues. The nanoparticles are adaptable for incorporation of a wide array of active compounds including pharmaceutical and nutraceutical compounds. The nanoparticles are particularly well-suited for incorporation and delivery of lipophilic compounds, for instance omega-3 fatty acid-containing compounds. The inventors further discovered means to enhance the antioxidant properties of the nanoparticles while increasing the overall stability of the nanoparticles and the active compound(s) incorporated therein. The nanoparticles can further increase the solubility and oral bioavailability of the incorporated active compound, thereby facilitating more effective dosage capabilities. The disclosure further provides methods of making the inventive nanoparticles, which can be adapted to provide an array of nanoparticle compositions. Also disclosed are disease treatment methods using the disclosed nanoparticles, which can be used to treat, for instance, cancer or tumors.

In one aspect, disclosed herein is a nanoparticle composition comprising 1) an active compound, or a pharmaceutically acceptable salt or prodrug thereof; 2) a pegylated vitamin E compound; and 3) at least one oil phase component.

In another aspect, disclosed herein is a nanoparticle composition comprising an active compound comprising a nucleobase analogue moiety covalently linked to an omega-3 polyunsaturated fatty acid moiety, or a pharmaceutically acceptable salt or prodrug thereof; a pegylated vitamin E compound; and at least one oil phase component.

In some embodiments, the nucleobase analogue moiety comprises gemcitabine. In some embodiments, the omega-3 polyunsaturated fatty acid moiety comprises docosahexaenoic acid. In some embodiments, the active compound comprises a compound having a Formula I:

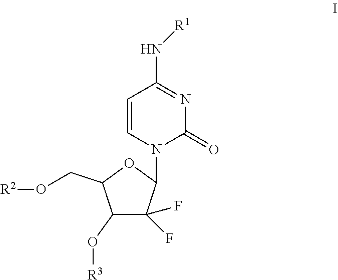

wherein R¹, R², and R³ are independently selected from hydrogen, halogen, hydroxyl, amino, thiol, thioalkyl, alkyl, alkenyl, alkynyl, haloalkyl, cycloalkyl, heterocycloalkyl, alkylaryl, aryl, alkylheteroaryl, heteroaryl, or omega-3 polyunsaturated fatty acid, any of which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro; wherein at least one of R¹, R², or R³ comprises an omega-3 polyunsaturated fatty acid.

In some embodiments, the active compound comprises 4-(N)-docosahexaenoyl 2′, 2′-difluorodeoxycytidine (DHA-dFdC). In some embodiments, the nanoparticle composition comprises the active compound in an amount up to about 1 weight percent (w/v), or up to about 0.65 weight percent (w/v). In some embodiments, the pegylated vitamin E compound comprises a polyethylene glycol having a molecular weight ranging from about 200 g/mol to about 6000 g/mol, wherein the polyethylene glycol is esterified to a vitamin E succinate. In some embodiments, the pegylated vitamin E compound comprises D-α-tocopherol polyethylene glycol 1000 succinate (TPGS). In some embodiments, the oil phase component comprises lecithin. In some embodiments, the composition further comprises an additional oil phase component, which can be a glycerol monostearate. In some embodiments, the composition further comprises an additional emulsifier, which can be a polysorbate. In some embodiments, the nanoparticle has an average diameter of 200 nm or less.

In another aspect, disclosed herein is a method of treating a subject with a disease comprising administering to the subject a therapeutically effective amount of a nanoparticle composition comprising an active compound comprising a nucleobase analogue moiety covalently linked to an omega-3 polyunsaturated fatty acid moiety, or a pharmaceutically acceptable salt or prodrug thereof; a pegylated vitamin E compound; and at least one oil phase component.

In some embodiments, the composition is administered parenterally, or can be administered orally. In some embodiments, the disease comprises a tumor. In some embodiments, the method reduces a rate of tumor growth. In some embodiments, the method increases tumor encapsulation. In some embodiments, the method increases the survival of tumor-bearing subject.

In yet another aspect, disclosed herein is a method of delivering an active compound to a biological cell comprising contacting the biological cell with a nanoparticle composition comprising the active compound comprising a nucleobase analogue moiety covalently linked to an omega-3 polyunsaturated fatty acid moiety, or a pharmaceutically acceptable salt or prodrug thereof; a pegylated vitamin E compound; and at least one oil phase component.

In yet another aspect, disclosed herein is a method of making a nanoparticle composition comprising combining an active compound comprising a nucleobase analogue moiety covalently linked to an omega-3 polyunsaturated fatty acid moiety, or a pharmaceutically acceptable salt or prodrug thereof; a pegylated vitamin E compound; and at least one oil phase component. In some embodiments, no organic solvents are used in the method.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same element(s) throughout the figures.

FIGS. 1A-1C are graphs and images showing effect of the amount of DHA-dFdC on the stability of the resultant DHA-dFdC-SLNs. After 6 days of storage at 4° C., the resultant DHA-dFdC-SLNs were analyzed for particle size (FIG. 1A), polydispersity index (FIG. 1B), and zeta potential (FIG. 1C). Data shown are mean±SD (n=3). FIG. 1D shows a representative particle size distribution curve of DHA-dFdC-SLNs prepared with 5.2 mg of DHA-dFdC. FIG. 1E shows a representative TEM image of DHA-dFdC-SLNs prepared with 5.2 mg of DHA-dFdC (bar=200 nm). FIG. 1F shows a representative gel permeation chromatograph of DHA-dFdC-SLNs prepared with 5.2 mg of DHA-dFdC. DHA-dFdC-SLNs were applied to a Sepharose 4B column, and the elution fraction was 0.5 mL.

FIGS. 2A-2C are graphs showing stability of DHA-dFdC and DHA-dFdC-SLNs as a lyophilized powder. On 0, 7 and 30 days after the DHA-dFdC-SLNs (made from 5.2 mg DHA-dFdC) were lyophilized and stored at room temperature, DHA-dFdC-SLNs were analyzed for particle size (FIG. 2A) and concentration of DHA-dFdC remaining in the DHA-dFdC-SLNs (FIG. 2B). FIG. 2C shows chemical stability of DHA-dFdC in a dry waxy solid that contains 5.047% (w/w) of vitamin E when stored at room temperature for 14 days. *** p<0.001. Data are mean±S.D. (n=3).

FIG. 3 is a graph showing the in vitro release profile of DHA-dFdC from DHA-dFdC-SLNs (made from 5.2 mg DHA-dFdC). Diffusion of DHA-dFdC (in Tween 20 micelles) across the dialysis membrane was measured as well. Data are mean±SD (n=3).

FIGS. 4A-4C are graphs showing cytotoxicity of DHA-dFdC-SLNs (made from 5.2 mg DHA-dFdC) in M-Wnt cells (FIG. 4A), B16-F10 cells (FIG. 4B), and TC-1 cells (FIG. 4C). Nanoparticles were incubated M-Wnt cells for 24 h, and with B16-F10 cells or TC-1 cells for 48 h. As controls, cells were also incubated with DHA-dFdC-free SLNs (“Blank-SLNs”), DHA-dFdC dissolved in DMSO (“DHA-dFdC”), or the equivalent concentration of DMSO (“DMSO”), or cell culture media alone. Data shown are mean±SD (n>3).

FIG. 5 is a graph showing plasma DHA-dFdC concentration (μg/mL) at different hourly (h) time points after DHA-dFdC-SLNs in suspension were intravenously injected into in C57BL/6 mice. The dose of DHA-dFdC was 2 mg per mouse. Data were fitted using the PKSolver, assuming a two-compartment model.

FIGS. 6A and 6B are graphs showing antitumor activity of DHA-dFdC-SLNs against B16-F10 tumors in mice. C57BL76 mice were subcutabeously (s.c.) injected with B16-F10 tumor on day 0. On day 7, mice were randomized into 5 groups (n=5-6) and intravenously (i.v.) injected with DHA-dFdC-SLNs, DHA-dFdC in vehicle, Blank-SLNs (DHA-dFdC-free SLNs) on days 7, 10, 13, and 16. The dose of DHA-dFdC was 50 mg/kg. After i.v. injection of treatments, tumor growth (FIG. 6A) and body weight change (FIG. 6B) were analyzed. As controls, one group of mice were left untreated. Data shown are mean±SEM. p<0.05; ^{a} DHA-dFdC-SLNs vs untreated; ^{b} DHA-dFdC-SLNs vs DHA-dFdC; DHA-dFdC-SLNs vs Blank-SLNs; ^{d} DHA-dFdC-SLNs vs vehicle.

FIGS. 7A-7G are a set of representative H&E images of B16-F10 tumors in C57BL76 mice i.v. injected with DHA-dFdC-SLNs, DHA-dFdC-free SLNs, DHA-dFdC in vehicle, vehicle alone, or untreated controls. Mice were euthanized on day 17 to collect tumor tissues. Tumor tissues of untreated (FIG. 7A), vehicle (FIG. 7B), and Blank-SLNs (FIG. 7C) groups are represented at a magnification 200×; while DHA-dFdC (FIGS. 7D and 7E) and DHA-dFdC-SLNs (FIGS. 7F and 7G) groups are represented by two different magnifications (100×(FIGS. 7D and 7F), 200×(FIGS. 7E and 7G)). The scale bars in the 100× images represent 100 μm, and that in the 200× images represent 50 μm. Black circles represent tumor area, dashed lines represent necrotic area, black arrows represent apoptotic cells, asterisk represent desmoplasia, white arrows represent blood vessel, times signs represent infiltration areas, black squares represent connective tissue areas, and stars represent necrotic cells.

FIGS. 8A-8G show the stability of DHA-dFdC-SLNs in simulated gastrointestinal fluids. DHA-dFdC-SLNs were incubated with simulated gastric fluid (SGF) (pH 1.2) or simulated intestinal fluid (SIF) (pH 6.8) at 37° C. Samples were collected at 0, 1, 2, 4 and 6 h, and particle diameter was measured (FIG. 8A). As a control, DHA-dFdC-SLNs were also incubated with PBS. Data are expressed as mean±SD (n=3). Shown in FIG. 8B-8G are representative TEM images of DHA-dFdC-SLNs incubated with PBS for 0 and 6 h (FIGS. 8B and 8C, respectively), SIF for 0 and 6 h (FIGS. 8D and 8E, respectively), or SGF for 0 and 6 h (FIGS. 8F and 8G, respectively). Bar=500 nm.

FIG. 9 is a graph showing in vitro release profiles of DHA-dFdC from DHA-dFdC-SLNs in simulated gastrointestinal fluids. As controls, the diffusion of DHA-dFdC (in DHA-dFdC-in Tween 20 micelles) across the dialysis membrane was also monitored. Data are mean±SD (n=3).

FIG. 10 is a graph showing plasma DHA-dFdC concentration-time curves after oral administration of DHA-dFdC-SLNs in suspension or DHA-dFdC in Tween 20-ethanol-water solution, or i.v. administration of DHA-dFdC-SLNs in suspension in healthy C57BL/6 mice. The dose of DHA-dFdC was 2 mg per mouse. Data are expressed as mean±S.D. (n=3).

FIG. 11 is a graph showing survival curves of B16-F10 tumor-bearing mice after oral treatment with DHA-dFdC-SLNs. Tumor cells were injected (s.c.) on day 0. On day 7, mice were randomized and orally gavaged with DHA-dFdC-SLNs in suspension or DHA-dFdC in a Tween 80-ethanol in water solution. As controls, mice received DHA-dFdC-free SLNs (blank-SLNs) or left untreated. * p<0.05, DHA-dFdC-SLNs vs. all other groups (Log-rank Mantel-Cox test. Data shown are mean±S.D. (n=7-8).

FIGS. 12A-12C are graphs showing representative particle size distribution curves of DHA-dFdC-SLNs prepared with different concentration of D-α-tocopherol polyethylene glycol 1000 succinate (TPGS): 0.4375 mg TPGS (FIG. 12A); 0.875 mg TPGS (FIG. 12B); 1.75 mg TPGS (FIG. 12C).

FIG. 13 is a representative TEM image of DHA-SLNs prepared with 5.31 mg of DHA (bar=100 nm).

FIG. 14 is a representative TEM image of docetaxel-SLNs prepared with 2.5 mg docetaxel (bar=100 nm).

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this invention belongs. The following definitions are provided for the full understanding of terms used in this specification.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular nanoparticle is disclosed and discussed and a number of modifications that can be made to the nanoparticle are discussed, specifically contemplated is each and every combination and permutation of the nanoparticle and the modifications that are possible unless specifically indicated to the contrary. Thus, if a class of nanoparticles A, B, and C are disclosed as well as a class of nanoparticles D, E, and F and an example of a combination nanoparticle, or, for example, a combination nanoparticle comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

Use of the phrase “and/or” indicates that any one or any combination of a list of options can be used. For example, “A, B, and/or C” means “A”, or “B”, or “C”, or “A and B”, or “A and C”, or “B and C”, or “A and B and C”.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., rheumatoid arthritis, cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of tumor growth. Therapeutically effective amounts of a given therapeutic agent will typically vary with respect to factors such as the type and severity of the disorder or disease being treated and the age, gender, weight, and general condition of the subject. Thus, it is not always possible to specify a quantified “therapeutically effective amount.” However, an appropriate “therapeutically effective amount” in any subject case may be determined by one of ordinary skill in the art using routine experimentation. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. It is understood that, unless specifically stated otherwise, a “therapeutically effective amount” of a therapeutic agent can also refer to an amount that is a prophylactically effective amount. In some instances, a desired biological or medical response is achieved following administration of multiple dosages of the composition to the subject over a period of days, weeks, or years.

“Treat,” “treating,” “treatment,” and grammatical variations thereof as used herein, include the administration of a composition with the intent or purpose of partially or completely, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, for example 1 to 3, 1 to 4, 1 to 5, 1 to 6, 1 to 7, 1 to 8, 1 to 9, 1 to 10, or 1 to 15 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms, for example, 2 to 5, 2 to 10, 2 to 15, or 2 to 20 carbon atoms, with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms, for example 2 to 5, 2 to 10, 2 to 15, or 2 to 20 carbon atoms, with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “carbonyl as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. Throughout this specification “C(O)” or “CO” is a short hand notation for C═O.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O—.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above. The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

“R′,” “R²,” “R³,” “Re,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

### Nanoparticle Compositions

It is understood that the nanoparticles of the present disclosure can be used in combination with the various compositions, methods, products, and applications disclosed herein.

The present disclosure addresses needs in the art by providing for nanoparticles having high incorporation efficiencies of pharmaceutical and/or nutraceutical compounds, and in which have slow release of such compounds when administered in vivo. The nanoparticles can incorporate high amounts of pharmaceutical and/or nutraceutical compounds for delivery at target tissues such as tumors while reducing delivery to nontarget tissues. The nanoparticles are primarily comprised of components which are generally recognized as safe (GRAS) components, thereby facilitating their use in pharmaceutical and/or nutraceutical applications. The nanoparticles can desirably increase the oral bioavailability of active compounds in vivo. In tumor models, drug-loaded nanoparticles can efficiently kill tumor cells and reduce tumor growth rates, or prolong the survival of tumor-bearing subjects. Very unexpectedly, some embodiments of the nanoparticles can facilitate tumor encapsulation with connective tissue, thereby slowing the growth rate of said tumor(s).

Solid Lipid Nanoparticles (SLNs) can be used as a delivery system for poorly water-soluble drugs (Feng, et al., Cancer Letters, 2013, 334, (2), 157-175; MuEller, et al., Euro. J. Pharma. Biopharma., 2000, 50, (1), 161-177; Geszke-Moritz, et al., Mater. Science Engineering, C 2016, 68, 982-994).

DHA-dFdC has excellent anti-tumor properties but is poorly water soluble. To improve water solubility and chemical stability of poorly water soluble compounds such as DHA-dFdC, disclosed herein is a novel solid lipid nanoparticle (SLN) formulation, which can contain DHA-dFdC (referred to herein as “DHA-dFdC-SLN”). The formulation further comprises a pegylated vitamin E compound, for instance D-α-tocopherol polyethylene glycol 1000 succinate (TPGS). TPGS is a water-soluble derivate of natural vitamin E, which is formed by esterification of vitamin E succinate with polyethylene glycol (PEG) (Zhang, et al., Biomat., 2012, 33, (19), 4889-4906). TPGS is used in pharmaceutical formulations as an emulsifier, solubilizer, absorption enhancer, permeation enhancer, and/or stabilizer (Zhang, et al., Biomat., 2012, 33, (19), 4889-4906; Mu, et al., J. Controlled Release, 2002, 80, (1), 129-144; Cho, et al., Intl. J. Nanomed., 2014, 9, 495; Muthu, et al., Intl. J. Pharma., 2011, 421, (2), 332-340). TPGS may also have stronger antioxidant activity than α-tocopherol or vitamin E (Carini, et al., Biochem. Pharma., 1990, 39, (10), 1597-1601; Anstee, et al., J. Hepatology, 2010, 53, (3), 542-550). Moreover, TPGS is a P-gp inhibitor and can help overcome multidrug resistance by tumor cells (Zhang, et al., Biomat., 2012, 33, (19), 4889-4906; Muthu, et al., Intl. J. Pharma., 2011, 421, (2), 332-340; Li, et al., Intl. J. Pharma., 2016, 512, (1), 262-272; Zhu, et al., Biomat., 2014, 35, (7), 2391-2400). Furthermore, TPGS can induce apoptosis and has synergic effects with certain cancer chemotherapeutics such as docetaxel, paclitaxel, and doxorubicin (Zhu, et al., Biomat., 2014, 35, (7), 2391-2400; Mi, et al., Biomat., 2011, 32, (16), 4058-4066; Youk, et al., J. Controlled Release, 2005, 107, (1), 43-52; Assanhou, et al., Biomat., 2015, 73, 284-295; Yu, et al., Acta Biomaterialia, 2015, 14, 115-124).

Disclosed herein is a nanoparticle composition comprising 1) an active compound, or a pharmaceutically acceptable salt or prodrug thereof; 2) a pegylated vitamin E compound; and 3) at least one oil phase component. By “active compound,” it is meant the compound can provide a therapeutic and/or nutraceutic benefit when administered to a subject without causing significant adverse effects at a dosage sufficient to achieve the therapeutic and/or nutraceutic benefit. The active compound can be any active compound capable of incorporation into the disclosed nanoparticles. Particularly desirable active compounds include hydrophobic and/or lipophilic active compounds, or generally poorly water soluble compounds. In some embodiments, the active compound can comprise an alkyl group, which can be an unsaturated alkyl group. In some embodiments, the alkyl group can comprise up to 50 carbon atoms. In some embodiments, the alkyl group can comprise up to 40 carbon atoms, up to 30 carbon atoms, up to 25 carbon atoms, up to 20 carbon atoms, up to 15 carbon atoms, or up to 10 carbon atoms. In some embodiments, the alkyl group can comprise from about 10 to about 50 carbon atoms, from about 10 to about 40 carbon atoms, from about 15 to about 30 carbon atoms, or from about 20 to about 25 carbon atoms. In some embodiments, the active compound can comprise a polyunsaturated fatty acid (PUFA) moiety.

Non-limiting examples of active compounds which can be incorporated into the disclosed nanoparticles include DHA-dFdC, docetaxel, retinoic acid, docosahexaenoic acid, vitamin A, atenelol, olmesartan medoxomil, mefenamic acid, diclofenac sodium, celecoxib, indomethacin, raloxifene, flutamide, tinidazole, clonazepam, ketoprofen, fluconazole, ibuprofen, moloxicam, prednisolone, aceclofenac, theophylline, cefixime, etoricoxib, telmisartan, nimesulide, irbesartan, cyclodextrins, bicalutamide, escitalopram oxalate, glipizide, dexamethasone, camphor, naproxen, proprionic acid, curcumin, ofloxacin, norfloxacin, ezetimide, indinavir, tolinolol, alendronate, acyclovir, diazepam, griseofulvin, albendazole, danazole, ketoconazole, itrconazole, atovaquone, troglitazone, valsartan, nimesulide, loratadine, felodipine, probucol, ubiquinone, cefixime frusemide, salicylic acid, hydrocholthiazide, nevirapine, clorazepate, rifampin, fentanyl, methoxyflurane, propanolol, propofol, thiopental, minoxidil, combinations thereof, as well as numerous other active compounds.

In some embodiments, the active compound comprises a nucleobase analogue moiety covalently linked to an omega-3 polyunsaturated fatty acid moiety, or a pharmaceutically acceptable salt or prodrug thereof. Active compounds comprising a nucleobase analogue moiety covalently linked to an omega-3 polyunsaturated fatty acid moiety are known and disclosed in US Patent Application Publication 2017/0157162, which is incorporated by reference herein in its entirety.

The nucleobase analogue moiety can be any chemical compound that can substitute for a normal nucleobase in nucleic acids. Nucleobases are nitrogen-containing biological compounds (e.g., nitrogenous bases) found within deoxyribonucleic acid (DNA), ribonucleic acid (RNA), nucleotides, and nucleosides. The primary nucleobases are cytosine, guanine, adenine, thymine, and uracil. Adenine and guanine belong to the double-ringed class of molecules called purines. Cytosine, thymine, and uracil are all pyrimidines. Modified nucleobases include hypoxanthine, xanthine, 7-methylguanine, 5,6-dihyfrouracil, 5-methylcytosine, cytarabine, 5-flurouracil, and 5-hydroxymethylcytosine.

Nucleobase analogues can comprise antimetabolites. An antimetabolite is a chemical that inhibits the use of a metabolite, which is another chemical that is part of normal metabolism. Such substances are often similar in structure to the metabolite they interfere with. The presence of antimetabolites can have toxic effects on cells, such as halting cell growth and cell division, so these compounds can be used as chemotherapy for cancer or to treat viral infections.

The compound formed when a nucleobase forms a glycosidic bond with the 1′ anomeric carbon of ribose or deoxyribose is called a nucleoside, and a nucleoside with one or more phosphate groups attached at the 5′ carbon is called a nucleotide. Thus, as used herein, nucleobase analogues include purine analogues, pyrimidine analogues, nucleoside analogues and nucleotide analogues.

Purine analogues are antimetabolites that mimic the structure of metabolic purines. Examples of purine analogues include, but are not limited to, azathioprine, mercaptopurine, thioguanine, flubarabine, pentostatin, and cladribine. Pyrimidine analogues are antimetabolites which mimic the structure of metabolic purines. Examples include, but are not limited to, 5-fluorouracil, floxuridine, cytosine arabinoside, and 6-azauracil.

Nucleoside analogues are molecules that act like the nucleosides in RNA or DNA synthesis. Once they are phosphorylated, they work as antimetabolites by being similar enough to nucleotides to be incorporated into growing RNA or DNA strands; but they can act as chain terminators. Example nucleoside analogues include, but are not limited to, (deoxy)adenosine analogues, (deoxy)cytidine analogues, (deoxy)guanosine analogues, (deoxy)thymidine analogues, (deoxy)uridine analogues, or combinations thereof. As used herein, for example, the term “(deoxy)adenosine” includes adenosine, deoxyadenosine, and combinations thereof. Other examples of nucleoside analogues include, but are not limited to, gemcitabine, fluororuacil, didanosine, vidarabine, cytarabine, emtricitabine, lamivudine, zalcitabine, abacavir, entecavir, stavudine, telbivudine, zidovudine, idoxuridine, trifluridine, apricitabine, or combinations thereof.

Polyunsaturated fatty acids (PUFAs) are fatty acids, e.g., a carboxylic acid with a long aliphatic tail, that contain more than one double bond in their backbone. Fatty acids have two ends, the carboxylic acid end, which is considered the beginning of the chain, thus “alpha”, and the methyl end, which is considered the tail of the chain, thus “omega”. The nomenclature of the fatty acid is taken from the location of the first double bond, counted from the methyl end, that is, the omega end. Therefore, omega-3 polyunsaturated fatty acids are those polyunsaturated fatty acids with a double bond at the third carbon atom from the end of the carbon chain. Examples of omega-3 PUFAs include, but are not limited to, alpha-linolenic acid (ALA), stearidonic acid (SDA), eicosatetroenoic acid (ETA), eicosapentaenoic acid (EPA), docosapentaenoic acid (DPA), and docosahexaenoic acid (DHA). In some embodiments, the omega-3 polyunsaturated fatty acids are chosen from docosahexaenoic acid, docosapentaenoic acid, eicosapentaenoic acid, alpha-linolenic acid, or any combination thereof. In other examples, the omega-3 polyunsaturated fatty acid is chosen from hexadecatrienoic acid, stearidonic acid, eicosatrienoic acid, eicosatetraenoic acid, heneicosapentaenoic acid, tetracosapentaenoic acid, and tetracosahexaenoic acid or any combination thereof.

Polyunsaturated fatty acids (PUFAs), including omega-3, omega-6 and omega-9 fatty acids, are vital to everyday life and function. For example, the beneficial effects of omega-3 fatty acids like all-cis-5,8,11,14,17-eicosapentaenoic acid (EPA) and all-cis-4,7,10,13,16,19-docosahexaenoic acid (DHA) on lowering serum triglycerides are well established. All-cis-9,12,15-octadecatrienoic acid (ALA) is the precursor essential fatty acid of EPA and DHA. All-cis-5,8,11,14-eicosatetraenoic acid (AA) and its precursors all-cis-6,9,12-octadecatrienoic acid (GLA) and all-cis-9,12-octadecadienoic acid (LA) have been shown to be beneficial to infants.

The omega-3 polyunsaturated fatty acid moiety can be synthetic or can be from (or derived from) natural sources, for instance from fish, algae, squid, yeast, and vegetable sources.

Various of these compounds are also known for other cardioprotective benefits such as preventing cardiac arrhythmias, stabilizing atherosclerotic plaques, reducing platelet aggregation, and reducing blood pressure. See e.g., Dyrberg et al., In: Omega-3 Fatty Acids: Prevention and Treatment of Vascular Disease. Kristensen et al., eds., Bi & Gi Publ., Verona-Springer-Verlag, London, pp. 217-26, 1995; O'Keefe and Harris, Am. J. Cardiology 2000, 85:1239-41; Radack et al., Arch Intern Med 151:1173-80, 1991; Harris, Curr Atheroscler Rep 7:375-80, 2005; Holub, CMAJ 166(5):608-15, 2002. Indeed, the American Heart Association has also reported that omega-3 fatty acids can reduce cardiovascular and heart disease risk. Other benefits of PUFAs are those related to the prevention and/or treatment of inflammation and neurodegenerative diseases, and to improved cognitive development. See e.g., Sugano and Michihiro, J. Oleo. Sci., 50(5):305-11, 2001.

In light of the health benefits of PUFAs, it is desirable to find new ways to deliver these and other beneficial materials to a subject. However, the hydrophobicity and oxidative stability (e.g., PUFAs are sensitive to oxidation) characteristics associated with many PUFAs creates significant challenges for incorporating them into compositions.

It is understood that reference herein to a particular PUFA bonded to the nucleobase analogue moiety can be a mixture of PUFA's. For example, certain fish oils, squid oils, seal oils, krill oils, rapeseed oil, flax, fungal oils, and algal oils can contain mixtures of omega-3, 6, and/or 9 fatty acids. These mixtures can be used and conjugated to nucleobase analogues, as disclosed herein.

In some embodiments, the omega-3 polyunsaturated acid moiety can be bonded directly to the nucleobase analogue moiety. For example, a compound as disclosed herein can be represented by the formula: CH₃—CH₂—CH═CH—Z—C(O)—XZ¹ wherein Z is a C₃-C₄₀ alkyl or alkenyl group comprising at least one double bond and Z¹ is nucleobase analogue moiety, and X is NH or O. In some embodiments, there is an additional ligand or spacer between the nucleobase analogue moiety and the omega-3 polyunsaturated acid moiety. Thus, Z¹ can be 1 to 10 atom linker and then nucleobase moiety.

In some examples, the nucleobase analogue comprises gemcitabine. Chemically, gemcitabine is a nucleoside analogue, specifically a deoxycytidine analogue, in which the hydrogen atoms on the 2′ carbon of deoxycytidine (a deoxyribonucleoside, a component of DNA) are replaced by fluorine atoms, as shown below.

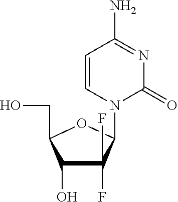

As with fluorouracil and other analogues of pyrimidines, the triphosphate analogue of gemcitabine replaces one of the building blocks of nucleic acids, in this case cytidine, during DNA replication. The process arrests tumor growth, as only one additional nucleoside can be attached to the “faulty” nucleoside, resulting in apoptosis. Another target of gemcitabine is the enzyme ribonucleotide reductase (RNR). The diphosphate analogue binds to RNR active site and inactivates the enzyme irreversibly. Once RNR is inhibited, the cell cannot produce the deoxyribonucleotides required for DNA replication and repair, and cell apoptosis is induced.

Compositions disclosed herein can contain compounds having Formula I:

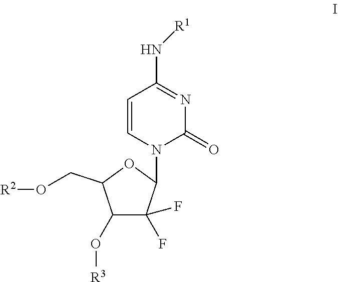

wherein R¹, R², and R³ are independently selected from hydrogen, halogen, hydroxyl, amino, thiol, thioalkyl, alkyl, alkenyl, alkynyl, haloalkyl, cycloalkyl, heterocycloalkyl, alkylaryl, aryl, alkylheteroaryl, heteroaryl, or omega-3 polyunsaturated fatty acid, any of which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro;

with proviso that at least one of R¹, R², or R³ comprises an omega-3 polyunsaturated fatty acid; or a pharmaceutically acceptable salt or prodrug thereof.

In some examples, the one or more omega-3 polyunsaturated fatty acid is bound directly to the gemcitabine-type compound. In some embodiments, there is an additional ligand or spacer between the one or more omega-3 polyunsaturated fatty acid and the gemcitabine-type compound.

In some examples, R¹, R² and R³ each independently comprise an omega-3 polyunsaturated fatty acid. In some examples, at least one of R¹, R², or R³ is CH₃—CH₂—CH═CH—Z—C(O)—X— wherein Z is a C₃-C₄₀ alkyl or alkenyl group comprising at least one double bond and X is NH or O. In other examples, at least one of R¹, R², or R³ is CH₃—CH₂—CH═CH—Z—C(O)—X-L- wherein Z is a C₃-C₄₀ alkyl or alkenyl group comprising at least one double bond, and L is a 1-10 atom linker, such as an alkyl or alkoxyl linker, and X is NH or O. In some examples, R¹ and R² each independently comprise an omega-3 polyunsaturated fatty acid while R³ does not comprise an omega-3 poly unsaturated fatty acid. In some examples R² and R³ each independently comprise an omega-3 polyunsaturated fatty acid, while R¹ does not comprise an omega-3 poly unsaturated fatty acid. In some examples R¹ and R³ each independently comprise an omega-3 polyunsaturated fatty acid, while R² does not comprise an omega-3 poly unsaturated fatty acid. In some examples R² comprises an omega-3 polyunsaturated fatty acid while R¹ and R³ do not comprise an omega-3 poly unsaturated fatty acid. In some examples R³ comprises an omega-3 polyunsaturated fatty acid, while R¹ and R² do no comprise an omega-3 poly unsaturated fatty acid. In some examples, R¹ comprises an omega-3 poly unsaturated fatty acid, while R² and R³ do not comprise an omega-3 poly unsaturated fatty acid.

In some examples of Formula I, where R² and R³ are hydrogen, the compounds have Formula IIA:

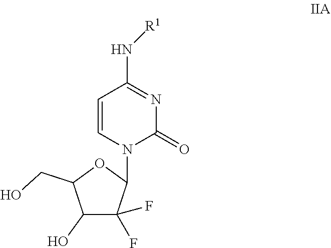

wherein R¹ comprises an omega-3-polyunsaturated acid which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro; or a pharmaceutically acceptable salt or prodrug thereof. For example, disclosed are compounds of Formula IIA where R¹ is CH₃—CH₂—CH═CH—Z—C(O)—X— wherein Z is a C₃-C₄₀ alkyl or alkenyl group comprising at least one double bond, and X is NH or O.

In some examples of Formula I, where R¹ and R³ are hydrogen, the compounds have Formula IIB:

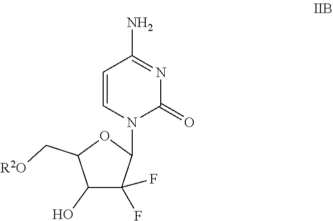

wherein R² comprises an omega-3-polyunsaturated acid which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro; or a pharmaceutically acceptable salt or prodrug thereof. For example, disclosed are compounds of Formula IIB where R² is CH₃—CH₂—CH═CH—Z—C(O)—X— wherein Z is a C₃-C₄₀ alkyl or alkenyl group comprising at least one double bond, and X is NH or O.

In some examples of Formula I, where R¹ and R² are hydrogen, the compounds have Formula IIC:

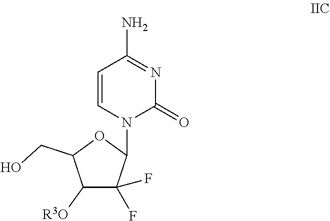

wherein R³ comprises an omega-3-polyunsaturated acid which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro; or a pharmaceutically acceptable salt or prodrug thereof. For example, disclosed are compounds of Formula IIC where R³ is CH₃—CH₂—CH═CH—Z—C(O)—X— wherein Z is a C₃-C₄₀ alkyl or alkenyl group comprising at least one double bond, and X is NH or O.

Also disclosed are compounds of formula I where more than one of R¹, R², and R³ comprise an omega-3-polyunsaturated acid which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro; or a pharmaceutically acceptable salt or prodrug thereof.

In some examples of Formula IIA, R¹ comprises docosahexaenoic acid, compounds are of Formula IIIA:

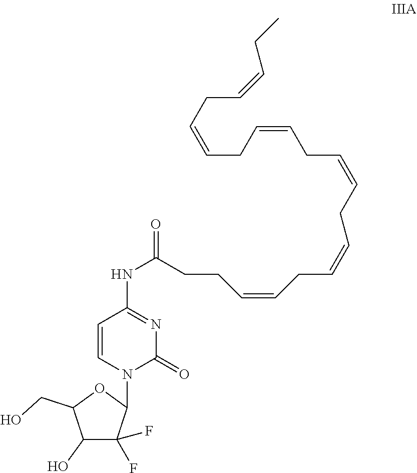 [SMILES: CC/C=C\C/C=C\C/C=C\C/C=C\C/C=C\C/C=C\CCC(=O)Nc1ccn(C2OC(CO)C(O)C2(F)F)c(=O)n1]

or a pharmaceutically acceptable salt or prodrug thereof. Additional examples of Formula IIB and IIC are Formulas IIIB and IIIC.

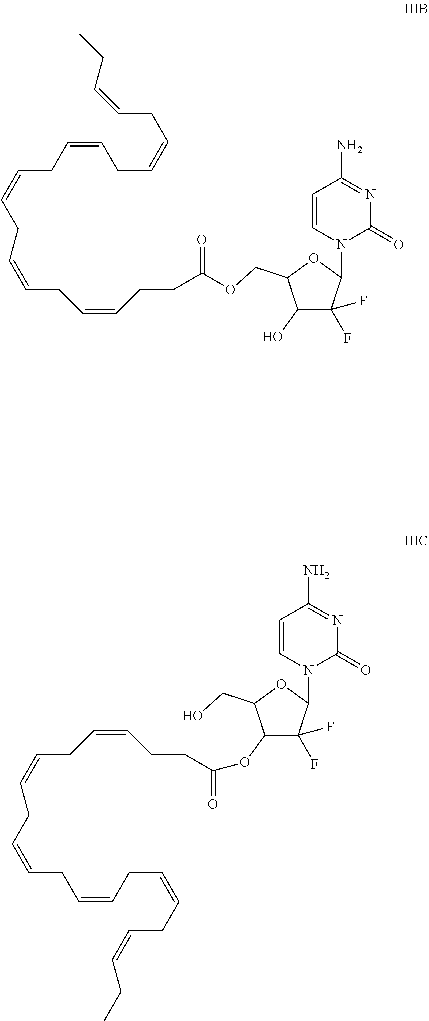 [SMILES: CC/C=C\C/C=C\C/C=C\C/C=C\C/C=C\C/C=C\CCC(=O)OCC1OC(n2ccc(N)nc2=O)C(F)(F)C1O ; CC/C=C\C/C=C\C/C=C\C/C=C\C/C=C\C/C=C\CCC(=O)OC1C(CO)OC(n2ccc(N)nc2=O)C1(F)F]

In some examples of Formula IIA, R¹ comprises eicosapentaenoic acid, compounds are of Formula IV:

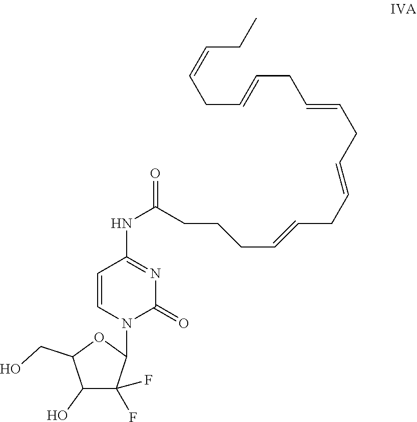 [SMILES: CC/C=C\C/C=C/C/C=C/C/C=C/C/C=C/CCCC(=O)Nc1ccn(C2OC(CO)C(O)C2(F)F)c(=O)n1]

or a pharmaceutically acceptable salt or prodrug thereof. Additional examples of Formula IIB and IIC are Formulas IVB and IVC.

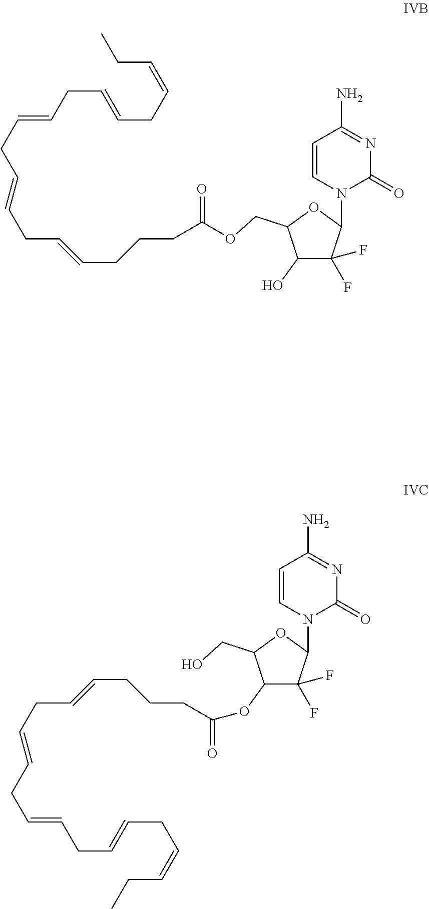 [SMILES: CC/C=C\C/C=C/C/C=C/C/C=C/C/C=C/CCCC(=O)OCC1OC(n2ccc(N)nc2=O)C(F)(F)C1O ; CC/C=C\C/C=C/C/C=C/C/C=C/C/C=C/CCCC(=O)OC1C(CO)OC(n2ccc(N)nc2=O)C1(F)F]

The compositions disclosed herein can also contain pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulphuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Compounds of Formulas I-IVC can be prepared beginning from gemcitabine HCl. For example, the hydroxyl groups of gemcitabine can be protected allowing for nucleophilic acyl substitution between the amine group of gemcitabine and the carboxylic acid group of the polyunsaturated fatty acid. Then the protecting groups can be removed to give the gemcitabine-polyunsaturated fatty acid compound.

The nanoparticle composition can comprise the active compound, for example an active compound comprising a nucleobase analogue moiety covalently linked to an omega-3 polyunsaturated fatty acid moiety, in an amount up to about 0.8 weight percent (w/v). As used herein, “weight percent (w/v)” refers to the percent of solute in a volume of solution (grams of solid/100 mL solution). In some embodiments, the nanoparticle composition can comprise the active compound in an amount up to about 0.75 weight percent (w/v), up to about 0.7 weight percent (w/v), up to about 0.65 weight percent (w/v), up to about 0.6 weight percent (w/v), up to about 0.52 weight percent (w/v), up to about 0.5 weight percent (w/v), up to about 0.4 weight percent (w/v), up to about 0.3 weight percent (w/v), up to about 0.2 weight percent (w/v), or up to about 0.1 weight percent (w/v). In some embodiments, the composition can comprise the active compound in an amount ranging from about 0.1 weight percent (w/v) to about 0.8 weight percent (w/v), from about 0.2 weight percent (w/v) to about 0.7 weight percent (w/v), or from about 0.3 weight percent (w/v) to about 0.52 weight percent (w/v).

The nanoparticle composition comprises a pegylated vitamin E compound. A “pegylated vitamin E compound” refers to one or more vitamin E-containing moieties covalently linked to one or more polyethylene glycol (PEG) moieties. A vitamin E moiety is a moiety comprised of one or more vitamin E compounds and can exhibit some of the characteristic properties of vitamin E such as antioxidant properties. Natural vitamin E compounds are mostly fat soluble and include the tocopherols and the tocotrienols. Both tocopherols and tocotrienols can have α, β, γ, or δ isoforms (e.g., α-tocopherol, γ-tocotrienol, etc.).

The polyethylene glycol (PEG) covalently linked to the vitamin E moiety is not particularly limited, and can range in size up to about 10,000 g/mol. In some embodiments, the PEG can have a size up to about 7,500 g/mol, up to about 5,000 g/mol, up to about 2,500 g/mol, up to about 2,000 g/mol, up to about 1,500 g/mol, or up to about 1,000 g/mol. In some embodiments, the PEG can have a size ranging from about 100 g/mol to about 10,000 g/mol, from about 200 g/mol to about 7,500 g/mol, from about 250 g/mol to about 6,000 g/mol, from about 400 g/mol to about 4,000 g/mol, from about 600 g/mol to about 3,000 g/mol, or from about 750 g/mol to about 2,000 g/mol. In some embodiments, the PEG can have a size of about 1,000 g/mol.

One particular form of a pegylated vitamin E compound is a tocopherol polyethylene glycol, which is commercially available in numerous forms. Often, a tocopherol polyethylene glycol is a water-soluble derivative of natural-source vitamin E prepared by esterifying D-α-tocopheryl acid succinate with polyethylene glycol (e.g., PEG-1000), and is commonly referred to as vitamin E TPGS or simply TPGS. Various forms of vitamin E TPGS are known and disclosed in U.S. Pat. Nos. 2,680,749 and 10,213,490, and in US Patent Application Publication 2007/0184117, each of which are incorporated by reference in their entireties. In some embodiments, the pegylated vitamin E compound comprises D-α-Tocopherol polyethylene glycol, or more particularly D-α-Tocopherol polyethylene glycol 1000 succinate.

The nanoparticle composition can comprise the pegylated vitamin E compound in an amount up to about 1.0 weight percent (w/v). In some embodiments, the nanoparticle composition can comprise the pegylated vitamin E compound in an amount up to about 0.9 weight percent (w/v), up to about 0.8 weight percent (w/v), up to about 0.5 weight percent (w/v), up to about 0.2 weight percent (w/v), up to about 0.175 weight percent (w/v), up to about 0.1 weight percent (w/v), up to about 0.75 weight percent (w/v), up to about 0.5 weight percent (w/v), up to about 0.25 weight percent (w/v), up to about 0.1 weight percent (w/v), up to about 0.09 weight percent (w/v), up to about 0.0875 weight percent (w/v), up to about 0.08 weight percent (w/v), up to about 0.07 weight percent (w/v), up to about 0.05 weight percent (w/v), up to about 0.044 weight percent (w/v), or up to about 0.02 weight percent (w/v). In some embodiments, the nanoparticle composition can comprise the pegylated vitamin E compound in an amount ranging from about 0.01 weight percent (w/v) to about 1 weight percent (w/v), from about 0.02 weight percent (w/v) to about 0.5 weight percent (w/v), from about 0.05 weight percent (w/v) to about 0.25 weight percent (w/v), or from about 0.0875 weight percent (w/v) to about 0.175 weight percent (w/v).

The ratio of the amount of the active compound to the amount of the pegylated vitamin E compound can be important, particularly for the overall size and morphology of the resultant nanoparticles made therefrom. In some embodiments, the active compound and the pegylated vitamin E compound can be present in the nanoparticle composition in a weight ratio ranging from about 1:10 to about 10:1. In some embodiments, the active compound and the pegylated vitamin E compound can be present in a weight ratio ranging from about 1:1 to about 8:1, from about 2:1 to about 6:1, or from about 3:1 to about 6:1.

The compositions further comprise at least one oil phase component. A wide array of oil phase components are compatible with the disclosed nanoparticles. The oil phase component can be branched or unbranched, and any given acyl chain can generally contain from 4 to 28 carbon atoms. Non-limiting examples of oil phase components include caproic acid, caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, linolenic acid, arachidonic acid, eicosapentaenoic acid, erucic acid, docosoahexanenoic acid, mono- and diglycerides, distilled monoglycerides, glycerol mono-stearates, sorbitan esters of hexitol anhydrides, sucrose esters, polyoxyethylene sorbitan esters of hexitol anhydrides, and chemical derivatives and combinations thereof.

In some embodiments, the oil phase component comprises a mixture of glycerophospholipids. The mixture of glycerophospholipids can be from a natural source or commercially produced. Such glycerophospholipids can include, but are not limited to, phosphatidylcholine, phosphatidylethanolamine, phosphatidylinositol, phosphatidylserine, and/or phosphatidic acid. In some embodiments, the oil phase component comprises lecithin. The lecithin can be animal-derived or plant derived, and can be from specific sources such as, without limitation, soybean, egg, milk, fish, rapeseed, cottonseed, and sunflower oil.

The nanoparticle composition can comprise the oil phase component in an amount up to about 10 weight percent (w/v). In some embodiments, the composition can comprise the oil phase component in an amount up to about 8 weight percent (w/v), up to about 6 weight percent (w/v), up to about 5 weight percent (w/v), up to about 4 weight percent (w/v), up to about 2 weight percent (w/v), up to about 1 weight percent (w/v), up to about 0.8 weight percent (w/v), up to about 0.75 weight percent (w/v), up to about 0.6 weight percent (w/v), up to about 0.5 weight percent (w/v), up to about 0.4 weight percent (w/v), up to about 0.25 weight percent (w/v), up to about 0.2 weight percent (w/v), or up to about 0.1 weight percent (w/v). In some embodiments, the nanoparticle composition can comprise the oil phase component in an amount ranging from about 0.01 weight percent (w/v) to about 10 weight percent (w/v), from about 0.05 weight percent (w/v) to about 5 weight percent (w/v), from about 0.1 weight percent (w/v) to about 1 weight percent (w/v), from about 0.25 weight percent (w/v) to about 0.75 weight percent (w/v), or from about 0.3 weight percent (w/v) to about 0.5 weight percent (w/v).

Optionally, the nanoparticle composition can comprise one or more additional emulsifiers. In some embodiments, the compositions can comprise one additional emulsifier, two additional emulsifiers, three additional emulsifiers, four additional emulsifiers, or five or more additional emulsifiers. The one or more additional emulsifiers can stabilize an emulsion by increasing its kinetic stability and is considered a surfactant or surface active agent. The one or more additional emulsifiers aids in emulsifying nonpolar, lipophilic, and/or hydrophobic components of the nanoparticle.

The one or more additional emulsifiers are not particularly limited and can be anionic emulsifiers, cationic emulsifiers, non-ionic emulsifiers or zwitterionic emulsifiers. The one or more additional emulsifiers can also be an additional oil phase component. In some embodiments, the one or more additional emulsifiers are selected from, as non-limiting examples, phosphatidylcholine; ethylene oxide copolymers, propylene oxide copolymers, poloxamers, sorbitan ethylene oxide/propylene oxide copolymers, polysorbate 20, polysorbate 60, polysorbate 80, sorbitan esters, span 20, span 40, span 60, span 80, alkylaryl polyether alcohol polymers, tyloxapol, bile salts, cholate, glycocholate, taurocholate, taurodeoxycholate; gemini surfactants and alcohols; modified starch or gum mixtures such as gum arabic, xanthan gum, guar gum, modified gum acacia, and/or an ester gum; acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum, C14-C22 fatty alcohols non-limiting examples of which are chosen from 1-tetradecanol (myristyl alcohol), 1-hexadecanol (cetyl alcohol), cis-9-hexadecen-1-ol (plamitoleyl alcohol), 1-octadecanol (stearyl alcohol), cis-9-octadecen-1-ol (oleyl alcohol), trans-9-octadecen-1-ol (elaidyl alcohol), 1-eicosanol (arachidyl alcohol), and 1-docosanol (behenyl alcohol). Further non-limiting examples of emulsifiers include esters of C14-C22 fatty alcohols and inorganic acids chosen from di-1-tetradecanyl phosphate (di-myristyl phosphate), di-1-hexadecanyl phosphate (di-cetyl phosphate), di-cis-9-hexadecen-1-yl phosphate (di-plamitoleyl phosphate), di-1-octadecanyl phosphate (di-stearyl phosphate), di-cis-9-octadecen-1-yl phosphate (di-oleyl phosphate), di-trans-9-octadecen-1-yl phosphate (di-elaidyl phosphate), di-1-eicosanyl phosphate (di-arachidyl phosphate), di-1-docosanyl phosphate (di-behenyl phosphate), 1-tetradecanyl sulfate (myristyl sulfate), 1-hexadecanyl sulfate (cetyl sulfate), cis-9-hexadecen-1-yl sulfate (plamitoleyl sulfate), 1-octadecanyl sulfate (stearyl sulfate), cis-9-octadecen-1-yl sulfate (oleyl sulfate), trans-9-octadecen-1-yl sulfate (elaidyl sulfate), 1-eicosanyl sulfate (arachidyl sulfate), and 1-docosanyl sulfate (behenyl sulfate), glyceryl monopalmitate, glyceryl monooleate, etc.; monostearin, monopalmitin, monoolein, Lactic acid esters of mono- and diglycerides of fatty acids, citric acid esters of mono- and diglycerides of fatty acids, mono- and diacetyl tartaric acid esters of mono- and diglycerides of fatty acids, sucrose esters of fatty acids, e.g., mono-, di- and triesters of sucrose with fatty acids; fatty acid esters of propane-1,2-diol such as 1-hydroxypropan-2-yl dodecanoate, 2-hydroxypropyl dodecanoate, propane-1,2-diyl didodecancoate, 1-hydroxypropan-2-yl tetradecanoate, 2-hydroxypropyl tetradecanoate, propane-1,2-diyl ditetradecancoate, 1-hydroxypropan-2-yl hexadecanoate, 2-hydroxypropyl hexadecanoate, and propane-1,2-diyl dihexadecancoate; polyoxyethylene glycol alkyl ethers; propylene glycol, 1,3-butylene glycol, glycerol, polyethylene glycols, fatty acid esters of sorbitan, and combinations thereof.

In some embodiments, the additional emulsifier comprises glycerol monostearate or polysorbate 20. In some embodiments, the composition comprises two additional emulsifiers, which can optionally comprise glycerol monostearate and polysorbate 20.

The nanoparticle composition can comprise an emulsifier in an amount up to about 10 weight percent (w/v). In some embodiments, the nanoparticle composition can comprise the additional emulsifier in an amount up to about 8 weight percent (w/v), up to about 6 weight percent (w/v), up to about 5 weight percent (w/v), up to about 4 weight percent (w/v), up to about 2 weight percent (w/v), up to about 1 weight percent (w/v), up to about 0.8 weight percent (w/v), up to about 0.75 weight percent (w/v), up to about 0.6 weight percent (w/v), up to about 0.5 weight percent (w/v), up to about 0.4 weight percent (w/v), up to about 0.25 weight percent (w/v), up to about 0.2 weight percent (w/v), or up to about 0.1 weight percent (w/v). In some embodiments, the nanoparticle composition can comprise the additional emulsifier in an amount ranging from about 0.001 weight percent (w/v) to about 10 weight percent (w/v), from about 0.005 weight percent (w/v) to about 5 weight percent (w/v), from about 0.01 weight percent (w/v) to about 1 weight percent (w/v), from about 0.01 weight percent (w/v) to about 0.1 weight percent (w/v), or from about 0.025 weight percent (w/v) to about 0.075 weight percent (w/v). In some additional embodiments, the nanoparticle composition can comprise the additional emulsifier in an amount ranging from about 0.1 weight percent (w/v) to about 10 weight percent (w/v), from about 0.5 weight percent (w/v) to about 5 weight percent (w/v), from about 0.75 weight percent (w/v) to about 2 weight percent (w/v), or from about 0.8 weight percent (w/v) to about 1.5 weight percent (w/v). Where more than one emulsifier is used (e.g., a first additional emulsifier and a second additional emulsifier), the amounts of the various additional emulsifiers within the compositions can be the same, overlapping, or different. As a non-limiting example, a first additional emulsifier can be present in an amount ranging from about 0.01 weight percent (w/v) to about 0.1 weight percent (w/v), whereas a second additional emulsifier can be present in an amount ranging from 0.5 weight percent (w/v) to about 5 weight percent (w/v).

The disclosed nanoparticles can be formed into a powder, pill, capsule, or other solid form. For example, the disclosed nanoparticles in solution can be lyophilized into a dry powder form. Inclusion of a lyoprotectant (e.g., sucrose, trehalose, glucose, fructose, sorbitol) can further stabilize the nanoparticles during lyophilization and in solid form. In such embodiments, it can be useful to describe the component ingredients in terms of weight percent based on solids, abbreviated herein as “weight percent (b.o.s.).” As used herein, the term “weight percent based on solids” or “weight percent (b.o.s.)” refers to the percentage of the solid component in the total solids consisting of the active compound, the pegylated vitamin E compound, and the oil phase component. Weight percent (b.o.s.) are disclosed without regard to solvent or optional solids (e.g., an additional emulsifier) which may or may not be present. Notably, usefulness of weight percent (b.o.s.) values are not limited to powder forms of the nanoparticles and are equally useful for volumetric solution formulations of the nanoparticles, or to refer to the components of the nanoparticles without regard to the physical formulation the nanoparticles are in.

The nanoparticles can comprise the active compound, for example an active compound comprising a nucleobase analogue moiety covalently linked to an omega-3 polyunsaturated fatty acid moiety, in an amount up to about 65 weight percent (b.o.s.). In some embodiments, the nanoparticles can comprise the active compound in an amount up to about 62 weight percent (b.o.s.), up to about 60 weight percent (b.o.s.), up to about 55 weight percent (b.o.s.), up to about 50 weight percent (b.o.s.), up to about 45 weight percent (b.o.s.), or up to about 40 weight percent (b.o.s.). In some embodiments, the nanoparticles can comprise the active compound in an amount ranging from about 35 weight percent (b.o.s.) to about 65 weight percent (b.o.s.), from about 40 weight percent (b.o.s.) to about 62 weight percent (b.o.s.), from about 50 weight percent (b.o.s.) to about 62 weight percent (b.o.s.), from about 50 weight percent (b.o.s.) to about 60 weight percent (b.o.s.), or from about 50 weight percent (b.o.s.) to about 55 weight percent (b.o.s.). In some embodiments, the nanoparticles can comprise the active compound in an amount of about 62 weight percent (b.o.s.), about 61 weight percent (b.o.s.), about 60 weight percent (b.o.s.), about 59 weight percent (b.o.s.), about 58 weight percent (b.o.s.), about 57 weight percent (b.o.s.), about 56 weight percent (b.o.s.), about 55 weight percent (b.o.s.), about 54 weight percent (b.o.s.), about 53 weight percent (b.o.s.), about 52 weight percent (b.o.s.), about 51 weight percent (b.o.s.), or about 50 weight percent (b.o.s.).

The nanoparticles can comprise the pegylated vitamin E compound in an amount up to about 20 weight percent (b.o.s.). In some embodiments, the nanoparticles can comprise the pegylated vitamin E compound in an amount up to about 15 weight percent (b.o.s.), up to about 10 weight percent (b.o.s.), up to about 9 weight percent (b.o.s.), up to about 8 weight percent (b.o.s.), up to about 7 weight percent (b.o.s.), up to about 6 weight percent (b.o.s.), up to about 5 weight percent (b.o.s.). In some embodiments, the nanoparticles can comprise the pegylated vitamin E compound in an amount ranging from about 1 weight percent (b.o.s.) to about 20 weight percent (b.o.s.), from about 2 weight percent (b.o.s.) to about 15 weight percent (b.o.s.), from about 3 weight percent (b.o.s.) to about 10 weight percent (b.o.s.), or from about 4 weight percent (b.o.s.) to about 9 weight percent (b.o.s.). The nanoparticles can comprise the oil phase component in an amount up to about 80 weight percent (b.o.s.). In some embodiments, the nanoparticles can comprise the oil phase component in an amount up to about 70 weight percent (b.o.s.), up to about 60 weight percent (b.o.s.), up to about 50 weight percent (b.o.s.), up to about 40 weight percent (b.o.s.), up to about 35 weight percent (b.o.s.), up to about 30 weight percent (b.o.s.), or up to about 25 weight percent (b.o.s.). In some embodiments, the nanoparticles can comprise the oil phase component in an amount ranging from about 10 weight percent (b.o.s.) to about 80 weight percent (b.o.s.), from about 15 weight percent (b.o.s.) to about 60 weight percent (b.o.s.), from about 20 weight percent (b.o.s.) to about 50 weight percent (b.o.s.), from about 25 weight percent (b.o.s.) to about 45 weight percent (b.o.s.), or from about 30 weight percent (b.o.s.) to about 40 weight percent (b.o.s.).

The disclosed nanoparticles can increase the water solubility of the active compound as compared to that compound's intrinsic water solubility (as a free compound). In some embodiments, the nanoparticles can increase the water solubility of the active compound, compared to the active compound's intrinsic water solubility, by at least 2-fold, at least 5-fold, at least 10-fold, at least 20-fold, at least 50-fold, at least 75-fold, at least 100-fold, at least 150-fold, or at least 200-fold or more.

The disclosed nanoparticles can further comprise an additional therapeutic or diagnostic agent. For instance, the therapeutic or diagnostic agent can be a small molecule or pharmaceutical, compound, amino acid or polypeptide, nucleic acid or polynucleotide, lipid, carbohydrate, glycolipid, polymer, etc. In some embodiments, the therapeutic or diagnostic agent is administrable to a subject.

Optionally, the nanoparticle can contain a targeting molecule to facilitate targeting of the nanoparticle to specific areas in vivo. The targeting molecule targets the nanoparticle to a particular tissue or cell type by specifically binding a ligand present in that tissue or cell type, or by being specifically altered by a cell, molecule, or condition present in that particular tissue or cell type. The targeting molecule can be any peptide, polypeptide, nucleic acid, polynucleotide, carbohydrate, lipid, small molecule, or synthetic molecule. For example, an antibody can target the nanoparticle to a cell type having a ligand to which the antibody specifically binds. Antibody targeting molecules can be polyclonal, monoclonal, fragments, recombinant, or single chain, many of which are commercially available or readily obtained using standard techniques. A targeting molecule can be attached to the nanoparticle via, for example, a hydrophobic linker which associates with the nanoparticle, or via linkage (e.g., covalently) with a surface molecule (e.g., an emulsifier).

The nanoparticle can have a diameter within the nanometer range (e.g., from 1 to 1,000 nm). In some embodiments, the nanoparticle has a diameter of 1,000 nm or less, 500 nm or less, 300 nm or less, or 200 nm or less. In some embodiments, the nanoparticle has a diameter from 10 nm to 500 nm, from 10 nm to 300 nm, from 10 nm to 250 nm, from 10 nm to 200 nm, or from 50 nm to 200 nm. Typically, nanoparticles formulated for ingestion or injection desirably have a diameter of 200 nm or less, which facilitates in vivo absorption and circulation of the nanoparticles. Nanoparticles formulated for non-ingested and non-injected administration (e.g., topical administration) can have a diameter larger than the nanometer range (e.g., from greater than 1,000 nm to 10,000 nm).

The nanoparticle can have a zeta potential of ±5 mV or more, as measured by dynamic light scattering methods. In some embodiments, the nanoparticle has a zeta potential of ±10 mV or more, ±15 mV or more, ±20 mV or more, ±25 mV or more, ±30 mV or more, ±40 mV or more, ±50 mV or more, ±60 mV or more, or ±70 mV or more, as measured by dynamic light scattering methods. In some embodiments, the nanoparticle has a zeta potential of about −20 to about −70 mV, about −30 to about −60 mV, or about −50 to about −60 mV.

The nanoparticles can have an efficient or advantageous encapsulation efficiency for the active compound. The term “encapsulation efficiency,” as used herein, refers to the percentage of active compound provided in a mixture with the pegylated vitamin E compound and the oil phase component that is ultimately encapsulated by nanoparticles formed therefrom. The nanoparticle can have an encapsulation efficiency of greater than 10% of the active compound, or greater than 25%, greater than 50%, greater than 75%, greater than 90%, greater than 95%, greater than 97%, or greater than 98% of the active compound.

The nanoparticle can have an advantageous burst release (e.g., an advantageous low extent of burst release), which is a percentage of active compound released from the nanoparticle in an aqueous solution (e.g., phosphate-buffered saline (PBS) at pH 7.4) over a period of time at 37° C. In some embodiments, the nanoparticle has a burst release of the active compound after 24 hours in an aqueous solution at 37° C. of 50% or less, 25% or less, 10% or less, or 5% or less.

In some embodiments, the nanoparticle can be formulated in a medicament. The nanoparticle can be formulated in any suitable medicament including, for example, but not limited to, solids, semi-solids, liquids, and gaseous (inhalant) dosage forms, such as tablets, pills, powders, liquid solutions or suspensions, suppositories, injectables, infusions, inhalants, hydrogels, topical gels, sprays, and the like. Optionally, the medicament comprises a pharmaceutically acceptable excipient. Optionally, the medicament comprises a therapeutically effective dose of the active compound.

### Methods of Treating

Also disclosed herein are methods of treating a subject with a disease comprising administering to the subject a therapeutically effective amount of a nanoparticle composition comprising an active compound (e.g. an active compound comprising a nucleobase analogue moiety covalently linked to an omega-3 polyunsaturated fatty acid moiety), or a pharmaceutically acceptable salt or prodrug thereof; a pegylated vitamin E compound; and at least one oil phase component. The nanoparticle can be any nanoparticle disclosed herein within the spirit of the invention.

Use of the nanoparticles in the disclosed methods is advantageous for a number of reasons, including the wide array of administration routes for which the nanoparticles are compatible, and the use of components generally recognized as safe (GRAS) to form the nanoparticles. As such, the nanoparticles can be administered in a number of ways to treat a variety of conditions and diseases. The nanoparticles are well-tolerated by administered subjects and can advantageously increase the bioavailability of the active compound compared to the free form of the active compound.

The administering step can include any method of introducing the particle into the subject appropriate for the particle formulation. In some embodiments, the composition is administered parenterally, or can be administered orally.

The administering step can include at least one, two, three, four, five, six, seven, eight, nine, or at least ten dosages. The administering step can be performed before the subject exhibits disease symptoms (e.g., prophylactically), or during or after disease symptoms occur. The administering step can be performed prior to, concurrent with, or subsequent to administration of other agents to the subject. The administering step can be performed with or without co-administration of additional agents (e.g., anti-cancer agents). In some embodiments, the amount of nanoparticles administered (and hence, the amount of active compound administered) is a therapeutically effective amount.

The amount of nanoparticles administered to the subject can be expressed in terms of a dosage amount per body weight, which can be calculated in terms of the nanoparticles or the active compound within the nanoparticles. The amount of the disclosed compositions administered to a subject will vary from subject to subject, depending on the nature of the disclosed compositions and/or formulations, the species, gender, age, weight and general condition of the subject, the mode of administration, and the like. Effective dosages and schedules for administering the compositions may be determined empirically, and making such determinations is within the skill in the art. The dosage ranges for the administration of the disclosed compositions are those large enough to produce the desired effect (e.g., to reduce tumor size). The dosage should not be so large as to outweigh benefits by causing adverse side effects, such as unwanted cross-reactions, anaphylactic reactions, and the like. The dosage can be adjusted by the individual clinician in the event of any counterindications. Generally, the disclosed compositions and/or formulations are administered to the subject at a dosage of active component(s) ranging from 0.1 μg/kg body weight to 100 g/kg body weight. In some embodiments, the disclosed compositions and/or formulations are administered to the subject at a dosage of active component(s) ranging from 1 μg/kg to 10 g/kg, from 10 μg/kg to 1 g/kg, from 10 μg/kg to 500 mg/kg, from 10 μg/kg to 100 mg/kg, from 10 μg/kg to 10 mg/kg, from 10 μg/kg to 1 mg/kg, from 10 μg/kg to 500 μg/kg, or from 10 μg/kg to 100 μg/kg body weight. Dosages above or below the range cited above may be administered to the individual subject if desired.

The subject can be any mammalian subject, for example a human, dog, cow, horse, mouse, rabbit, etc. In some embodiments, the subject is a primate, particularly a human. The subject can be a male or female of any age, race, creed, ethnicity, socio-economic status, or other general classifiers.

In some embodiments, the disease is a cell-cycle regulation disorder. In some embodiments, the disease comprises a tumor or cancer. Non-limiting examples of cancers include Acute granulocytic leukemia, Acute lymphocytic leukemia, Acute myelogenous leukemia (AML), Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma, Bile duct cancer, Bladder cancer, Bone cancer Bone marrow cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia (CLL), Chronic myelogenous leukemia (CML), Colon cancer, Colorectal cancer, Craniopharyngioma, Cutaneous lymphoma, Cutaneous melanoma, Diffuse astrocytoma, Ductal carcinoma in situ (DCIS), Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors (GIST), Germ cell tumor, Gestational Trophoblastic Disease (GTD), Glioblastoma multiforme (GBM), Glioma, Hairy cell leukemia, Head and neck cancer, Hemangioendothelioma, Hodgkin's lymphoma, Hypopharyngeal cancer, Infiltrating ductal carcinoma (IDC), Infiltrating lobular carcinoma (ILC), Inflammatory breast cancer (IBC), Intestinal Cancer, Intrahepatic bile duct cancer, Invasive/infiltrating breast cancer, Islet cell cancer, Jaw/oral cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Mycosis Fungoides, Myelodysplastic Syndrome, Nasal cavity cancer, Nasopharyngeal cancer, Neck cancer, Neuroblastoma, Neuroendocrine tumors (NETs), Non-Hodgkin's lymphoma, Non-small cell lung cancer (NSCLC), Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system (CNS) lymphoma, Prostate cancer, Rectal cancer, Renal cell carcinoma, Renal pelvis cancer, Rhabdomyosarcoma, Salivary gland cancer, Sarcoma, Sinus cancer, Skin cancer, Small cell lung cancer (SCLC), Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma, Testicular cancer, Throat cancer, Thymoma/thymic carcinoma, Thyroid cancer, Tongue cancer, Tonsil cancer, Transitional cell cancer, Transitional cell cancer, Triple-negative breast cancer, Tubal cancer, Tubular carcinoma, Ureteral cancer, Urethral cancer, Uterine adenocarcinoma, Uterine cancer, Uterine sarcoma, Vaginal cancer, Vulvar cancer, Wilms tumor, Waldenstrom macroglobulinemia, etc., and combinations thereof.

Administration of the disclosed nanoparticles can be used to deliver an active compound (e.g., an active compound comprising a nucleobase analogue moiety covalently linked to an omega-3 polyunsaturated fatty acid moiety) to a tumor or a tumor environment. In some embodiments, the method reduces a rate of tumor growth. In some embodiments, the method reduces the size of a tumor. In some embodiments, the method reduces the metastasis of a tumor. In some embodiments, the method reduces recurrence of a tumor. In some embodiments, the method increases the survival of a subject having a tumor (e.g., a tumor-bearing mouse or a human tumor patient). In some embodiments, the methods reduce the release of the active compound in non-target tissues (e.g., non-cancerous tissues in a method to treat cancer). In some embodiments, the methods increase the bioavailability of the active compound. In some embodiments, the methods reduce the toxicity of the active compound.

One surprising finding includes that the disclosed method can, in some embodiments, increase the amount of fibrous connective tissue within a tumor microenvironment. The tumor microenvironment includes the tumor and surrounding tissue which can affect, or be affected by, the tumor. Increasing amounts of fibrous connective tissue surrounding a tumor, sometimes referred to as a fibrous connective tissue capsule, can restrict or impede the growth of a tumor encapsulated therein. This phenomenon can be referred to as “tumor encapsulation” and can produce therapeutically beneficial results for a cancer patient. Thus, as used herein, increasing the amount of “tumor encapsulation” refers to increasing the amount of fibrous connective tissue within a tumor microenvironment surrounding a tumor.

Results obtained after administration of the nanoparticles can be compared to a control. Optionally, the control is a biological sample. Alternatively, the control can be a collection of values used as a standard applied to one or more subjects (e.g., a general number or average that is known and not identified in the method using a sample). In some embodiments, the control comprises a blood, plasma, serum, mucosal, or gastrointestinal fluid sample obtained from the subject prior to the administration step (e.g., a baseline sample). In some embodiments, the control can comprise a biological sample of the subject known not to be or suspected not to be cancerous.

The nanoparticles can optionally be administered in a medicament. The medicament can further comprise a pharmaceutically acceptable excipient. Optionally, the medicament comprises a therapeutically effective dose of an active compound.

In yet another aspect, disclosed herein is a method of delivering an active compound to a biological cell comprising contacting the biological cell with a nanoparticle composition comprising the active compound (e.g., an active compound comprising a nucleobase analogue moiety covalently linked to an omega-3 polyunsaturated fatty acid moiety), or a pharmaceutically acceptable salt or prodrug thereof; a pegylated vitamin E compound; and at least one oil phase component. The nanoparticle can be any herein disclosed nanoparticle within the spirit of the invention. In some embodiments, contacting the nanoparticle with the biological cell releases the active compound from the nanoparticle. In some embodiments, contacting the nanoparticle with the biological cell results in death of the cell. In some embodiments, the biological cell is a cancerous cell.

### Methods of Making Nanoparticles

Also disclosed herein are methods to make the disclosed nanoparticles. The methods are advantageous at least because they result in particles having 1) high active compound encapsulation efficiencies, 2) reduced burst release of the active compound, 3) small diameters (e.g., about 50-200 nm), which are ideal for targeted delivery of agents to, e.g., tumors, 4) negative zeta potential, indicating high stability and less toxicity in vitro and in vivo, and 5) increased oral bioavailability of the active compound as compared to the free form of the active compound.

Thus, disclosed herein is a method of making a nanoparticle comprising combining an active compound (e.g., an active compound comprising a nucleobase analogue moiety covalently linked to an omega-3 polyunsaturated fatty acid moiety), or a pharmaceutically acceptable salt or prodrug thereof; a pegylated vitamin E compound; and at least one oil phase component. In some embodiments, no organic solvents are used in the method. In some embodiments, the pegylated vitamin E compound and the at least one oil phase component are generally recognized as safe (GRAS) components. In some embodiments, the methods further comprise combining one or more additional emulsifier(s). In some embodiments, the one or more additional emulsifier(s) are generally recognized as safe (GRAS) components.

The combining steps can be performed by any method useful to combine the recited components. For example, the components can be combined by adding, pouring, titrating, mixing, dissolving, injecting, etc. A first component can be combined by addition to a second component, or vice versa. Alternatively, numerous components can be combined with each other or into another component. Optionally, any one or more combining steps are performed while stirring or mixing the components (e.g., stirring via a stir bar at 100 rpm in a fume or chemical hood).

In some or further embodiments, the method can include collecting or concentrating the nanoparticles. The nanoparticles can be collected by, for example, centrifugation or ultrafiltration. Nanoparticles can be washed and resuspended in desirable buffered solutions at desirable concentrations. In some embodiments, the nanoparticles can be lyophilized into a dry powder, or a wet powder, form.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. A Solid Lipid Nanoparticle Formulation of 4-(N)-docosahexaenoyl 2′, 2′-difluorodeoxycytidine Having Potent, Broad Spectrum Antitumor Activity

Disclosed in this example is a solid lipid nanoparticle (SLN) formulation comprising DHA-dFdC with improved apparent aqueous solubility and chemical stability. SLNs further comprised lecithin/glycerol monostearate-in-water emulsions emulsified with D-α-tocopherol polyethylene glycol 1000 succinate (TPGS) and Tween 20. The resultant DHA-dFdC-SLNs were 102.2±7.3 nm in diameter and increased the solubility of DHA-dFdC in water to at least 5.2 mg/mL, more than 200-fold higher than its intrinsic water solubility. As a comparison, the waxy solid of DHA-dFdC, even in the presence of vitamin E as an antioxidant, was unstable when stored at room temperature. However, after one-month of storage at the same condition, DHA-dFdC in lyophilized DHA-dFdC-SLNs powder did not significantly degrade. DHA-dFdC-SLNs also showed increased cytotoxicity against certain tumor cells than DHA-dFdC. Plasma concentration of DHA-dFdC in mice intravenously injected with DHA-dFdC-SLNs in dispersion followed a bi-exponential model, with a half-life of ˜44 h. In mice with pre-established B16-F10 murine melanoma, DHA-dFdC-SLNs were significantly more effective than free DHA-dFdC in controlling the tumor growth. In addition, histology results revealed a high level of apoptosis and tumor encapsulation in tumors in mice treated with DHA-dFdC-SLNs.

### Materials and Methods

### List of Non-Standard Abbreviations

DHA-dFdC, 4-(N)-docosahexaenoyl 2′, 2′-difluorodeoxycytidine; PUFA, polyunsaturated fatty acid; dFdC, 2′, 2′-difluorodeoxycytidine; IV, intravenous; DHA, docosahexaenoic acid; SLNs, solid lipid nanoparticles, GMS, glycerol monostearate; TPGS, D-α-Tocopherol polyethylene glycol 1000 succinate or vitamin E TGPS.

Materials and Cell Lines.

Mannitol, Tween 20, glycerol monostearate (GMS), D-α-tocopherol polyethylene glycol 1000 succinate (TPGS), 3-(4,5-dimethylthiazol-2-yl)-2,5-diphenyltetrazolium bromide (MTT), Tween 80, mannitol, and sucrose were from Sigma-Aldrich (St. Louis, Mo.). Gemcitabine HCl was from Biotang, Inc. (Lexington, Mass.). Soy lecithin was from Alfa Aesar (Ward Hill, Mass.). Ethyl acetate (EtOAc), dimethyl sulfoxide, tetrahydrofuran (HPLC-grade), isopropanol, and methanol (HPLC-grade) were from Thermo Fisher (Waltham, Mass.). Float-A-Lyzer®G2 dialysis device (MWC 50 kD) was from Spectrum Inc. (New Brunswick, N.J.)

B16-F10 murine melanoma cell and TC-1 murine lung cancer cell lines were from the American Type Culture Collection (Manassas, Va.). M-Wnt cells (murine mammary gland cell lines) were from Dr. Stephen D. Hursting's lab at The University of North Carolina, Chapel Hill. B16-F10 and TC-1 cells were grown in DMEM and RPMI 1640, respectively (Invitrogen, Carlsbad, Calif.). M-Wnt cells were grown in a similar medium as TC-1, with an additional supplement of 1% Glutamax (GlutaMAXTMSupplement, Gibco®). All media were supplemented with 10% (v/v) fetal bovine serum (FBS), 100 U/mL of penicillin, and 100 μg/mL of streptomycin, all from Invitrogen (Carlsbad, Calif.).

Preparation of DHA-dFdC-SLNs.

DHA-dFdC was synthesized following a previously reported conjugation scheme (Naguib, et al., Neoplasia, 2016, 18, (1), 33-48). The purity of the resultant DHA-dFdC was confirmed by NMR and Mass Spectrum. Solid Lipid Nanoparticles (SLNs) were prepared by, as an example, combining 3.5 mg of soy lecithin, 0.5 mg of glycerol monostearate (GMS), and 0.875 mg D-α-tocopherol polyethylene glycol 1000 succinate (TPGS) into a glass vial. 800 μL de-ionized and filtered (0.22 μm) water (80° C.) were added into the lecithin/GMS/TPGS mixture, which was then vortexed and sonicated for 3 min until a homogenous slurry was formed. The mixture was maintained on an 80° C. hot plate surface for 5 min. A solution of Tween 20 (55 mg in 1 ml of water) was prepared, and 200 μL of this solution was added drop wise into the mixture to reach a final concentration of 1% (v/v). The resultant emulsions were allowed to cool to room temperature while stirring to form SLNs. To incorporate DHA-dFdC into the SLNs, DHA-dFdC at various amounts (for example, 5.2, 8.3, 9.8, or 14.3 mg) were added into the lecithin/GMS/TPGS mixture before the addition of water. Preparation of DHA-dFdC-free SLNs followed the same procedure but without the addition of DHA-dFdC.

Short-Term Stability Study.

Stability of DHA-dFdC-SLNs prepared with 0, 5.2, 8.3, 9.8, or 14.3 mg of DHA-dFdC was evaluated at 4° C. for 6 days. Size and zeta potential of resultant SLNs were measured using a Malvern Zetasizer Nano ZS (Westborough, Mass.).

Transmission Electron Microscopy (TEM).

Size and morphology of DHA-dFdC-SLNs were examined using a transmission electron microscope available in the Institute for Cellular and Molecular Biology Microscope and Imaging Facility at The University of Texas at Austin. The carbon film-coated copper grid was glow discharged for 2 min. A sample of 10 μL of DHA-dFdC-SLNs suspended in water was deposited on the grid and left to stand for 1 min. Excess sample was removed with a filter paper. One drop of 1% uranyl acetate was added on the grid for 30 s. The sample was then observed under the TEM after removing the excess uranyl acetate fluid with filter paper (Zhu et al., Bioconjugate Chem., 2012, 23(5):966-980).

Encapsulation Efficiency (EE).

The encapsulation efficiency of DHA-dFdC in SLNs was determined by an ultrafiltration method. 1 mL of DHA-dFdC-SLNs was added into an ultrafiltration centrifuge tube (30 kD, Amicon Ultra-4, Millipore) and centrifuged at 2844 rcf for 10 min. 100 μl of the filtrate solution was taken from the bottom part of the ultrafiltration centrifuge tube to measure DHA-dFdC concentration by high performance liquid chromatography (HPLC). To corroborate the detection method, the remaining suspension (about 50 μl) in the ultrafiltration centrifuge tube was re-dissolved with 950 μl water to extract the DHA-dFdC, according the procedure previously described.

Gel Permeation Chromatography (GPC).

To separate potential micelles from DHA-dFdC-SLNs, GPC was performed using a 6 mm×30 cm Sepharose® 4B column (Sloat et al, Intl. J. Pharma., 2011; 409(1):278-288). Samples (100 μL) were applied into the column and eluted with de-ionized and filtered (0.22 μm) water. Elution fractions of 500 μL were collected. Particle concentration (measured as kilo counts per second; Kcps) in each fraction was measured using a Malvern Zetasizer Nano ZS, and the concentration of DHA-dFdC in each fraction was determined using HPLC after extraction.

Lyophilization of the DHF-dFdC-SLNs and their Stability in Lyophilized Powder.

A 30% (w/v) stock solution of sucrose as lyoprotectant was prepared with de-ionized and filtered (0.2 μm) water. 900 μL DHA-dFdC-SLNs in water suspension was mixed with 100 μL sucrose solution to obtain a final suspension having 3% (w/v) sucrose. DHA-dFdC-SLNs in suspension were stored at −20° C. for 30 min, transferred to −80° C. for 60 min, and finally transferred to a VirTis Advantage bench top tray lyophilizer (The VirTis Company, Inc. Gardiner, N.Y.). Lyophilization was performed over 72 hours (h) at pressure less than 200 mTorr under nitrogen atmosphere. The shelf temperature was gradually ramped from −40° C. to 26° C. After lyophilization, samples were sealed and stored in a desiccator at room temperature, protected from light.

To evaluate the physical and chemical stability of DHA-dFdC-SLNs in lyophilized powder, DHA-dFdC was extracted from the powder 0, 7, and 30 days post-storage. Lyophilized samples were reconstituted in 1 mL de-ionized and filtered (0.2 μm) water. The reconstituted DHA-dFdC-SLN suspension (100 μL) was mixed with 100 μL isopropanol, vortexed for 30 s, and maintained at room temperature for 5 min. 600 μl ethyl acetate was added, and the sample was vortexed for 30 s and centrifuged at 11,000 rcf for 20 min. The supernatant was collected into a glass vial and evaporated under nitrogen. The sample was re-dissolved in 100 μL of THF, and concentration was measured by HPLC.

As a control, DHA-dFdC was dissolved in ethanol and mixed with vitamin E at a final concentration of 5.047% (w/w) (Naguib et al., Neoplasia, 2016; 18(1):33-48). The solution was dried under nitrogen, sealed, and stored at room temperature, protected from light, and the content of DHA-dFdC was measured at various time points

In Vitro Stability of DHA-dFdC-SLNs in Simulated Biological Media.

To evaluate stability of DHA-dFdC-SLNs in simulated biological media, SLNs in suspension were diluted in phosphate-buffered saline (PBS, 10 min, pH 7.4) with 10% FBS (v/v) and incubated at 37° C. in a MaxQ 4000 Floor Shaker Incubator (Thermo Fisher Scientific, 100 rpm) for 18 h. Particle size was measured at varying time points using a Malvern Zetasizer.

In Vitro Release of DHA-dFdC from DHA-dFdC-SLNs.

The release profile of DHA-dFdC from SLNs was evaluated by suspending DHA-dFdC-SLNs at an example concentration of 127 μg/mL in release medium (1% (w/v) Tween 20 in PBS), which were then placed into a 1 mL cellulose ester dialysis tube (MWC 50,000) from Spectrum Chemicals & Laboratory Products (New Brunswick, N.J.). The dialysis tube was placed into a plastic conical tube containing 13 mL release medium to create sink conditions, which was incubated in a MaxQ 5000 Floor Shaker Incubator at 37° C. and 100 rpm for 8 h. At predetermined time points, 200 μL release medium was withdrawn and replaced with 200 μL of fresh release medium. As a control, the diffusion of DHA-dFdC dissolved in a Tween 20 solution (127 μL g/mL of DHA-dFdC in 1% aqueous Tween 20 solution) across the dialysis membrane was also measured. Concentration of DHA-dFdC was determined by HPLC.

HPLC.

HPLC analysis of DHA-dFdC was performed using an Agilent Infinity 1260 (Santa Clara, Calif.) with a RP-C18 column (Zorbax Eclipse, 5 μm, 4.5 mm×150 mm, Santa Clara, Calif.). The mobile phase was methanol and water (90:10, v/v). The flow rate was 1.0 ml/min, and the detection wavelength and injection volume were 248 nm and 5 μL, respectively (Naguib et al., Neoplasia, 2016; 18(1):33-48).

In Vitro Cytotoxicity Assay.

Cytotoxicity of DHA-dFdC-SLNs was evaluated in TC-1, B16-F10, and M-Wnt cells. Cells were seeded into 96-well plates (4000 cells/well for TC-1 and B16-F10 cells, 1000 cells/well for M-Wnt cells) and incubated at 37° C., 5% CO₂ overnight. Cells were treated with various concentrations of DHA-dFdC, DHA-dFdC-SLNs, DHA-dFdC-free SLNs, or dimethyl sulfoxide (DMSO) for up to 48 h. As a control, cells were treated with fresh medium. Cell survival was determined using an MTT assay (Naguib et al., Mol. Pharma., 2014; 11(4):1239-1249). DHA-dFdC was dissolved in DMSO and diluted with cell culture media, whereas DHA-dFdC-SLNs and DHA-dFdC-free SLNs were dispersed directly in cell culture media.

Plasma pharmacokinetics (PK) of DHA-dFdC in DHA-dFdC-SLNs. The animal protocol was approved by the Institutional Animal Care and Use Committee at The University of Texas at Austin. To evaluate PK parameters, healthy female C57BL/6 mice (6-8 weeks, Charles River Laboratories, Wilmington, Mass.) were injected intravenously with DHA-dFdC-SLNs dispersed in sterile mannitol 5% (w/v) at dose of 2 mg of DHA-dFdC per mouse. Mice were euthanized at various time points (0.25, 0.5, 1, 2, 4, 8, 24, and 48 h). Blood was collected into heparin-coated tubes, which were then centrifuged at 13000 rcf for 20 min to isolate plasma. 200 μL plasma was mixed with 200 μL isopropanol and 200 μL cold PBS. The mixture was vortexed and incubated at 4° C. for 5 min. Following incubation, 1000 μL of ethyl acetate was added, and the mixture was vortexed for 5 min, and followed by centrifugation at 18,000 rcf for 5 min. The supernatant was collected and dried under nitrogen gas. Finally, the residue was re-dissolved in 100 μL THF, which was then analyzed using HPLC (Naguib et al., Neoplasia, 2016; 18(1):33-48). As an internal control, 4-(N)-stearoyl dFdC synthesized by conjugating stearate and dFdC on its 4-(N) position was added in the samples before extraction (Sloat et al., Intl. J. Pharma., 2011; 409(1):278-288). Data were analyzed using the PK Solver®, assuming a two-compartmental model (Zhang et al., Comp. Meth. Prog. Biomed., 2010; 99(3):306-314).

Evaluation of the Antitumor Activity of DHA-dFdC-SLNs in a Mouse Model.

Female C57BL/6 mice (18-20 g, 6-8 weeks) were subcutaneously (s.c.) injected with B16-F10 (5×10⁵ cells/mouse) in the right flank on day 0. Seven days later, mice were randomized in 5 groups (n=5-6) and i.v. injected with DHA-dFdC (1 mg/mouse, equivalent to 50 mg/kg) dissolved a vehicle solution (Tween 80 (10%, w/v), ethanol (5.2%, v/v), and mannitol (5%, w/v) in water), the vehicle solution (as a control) (Naguib et al., Neoplasia, 2016; 18(1):33-48; Valdes et al., Pharma. Res., 2017; 34(6):1224-1232), DHA-dFdC-SLNs (equivalent to 1 mg of DHA-dFdC/mouse), or the equivalent dose of DHA-dFdC-free SLNs; both SLNs were dispersed in sterile mannitol 5%, (w/v). As a control, one group of mice were left untreated. Treatments were repeated every 3 days for a total of 4 times. Mouse health and tumor growth were monitored daily. Tumor size was measured 2-3 times a week, and tumor volume was calculated as: volume (mm³)=(length×width²)/2. Mice were euthanized 17 days after B16-F10 cell injection, and tumor tissues were collected for histology study. For untreated mice, the length of some of the tumors reached 15 mm before day 17 and had to be euthanized earlier.

Histology.

Tumor tissues were fixed in formalin, embedded, and stained with hematoxylin and eosin (H&E) in the Histological and Tissue Analysis Facility in the Dell Pediatric Research Institute at The University of Texas at Austin.

Data Analysis.

Statistical analyses were completed by one-way ANOVA followed by a Bonferroni post hoc test. A p value of 0.05 (two-tail) was considered significant. Most of the analyses were performed with GraphPad Prism (GraphPad Software, Inc., La Jolla, Calif.). PK parameters were obtained using PK Solver (Zhang et al., Comp. Meth. Prog. Biomed., 2010; 99(3):306-314).

### Results and Discussion

Preparation and characterization of DHA-dFdC-SLNs. DHA-dFdC is a lipophilic compound with potent antitumor activity against various cancer cell lines in culture (e.g. pancreatic cancer, leukemia, kidney cancer) and in mouse models of pancreatic cancer and leukemia (Naguib et al., Neoplasia, 2016; 18(1):33-48; Valdes et al., Pharma. Res., 2017; 34(6):1224-1232). However, the solubility and stability of this compound need to be improved (Naguib et al., Neoplasia, 2016; 18(1):33-48). Disclosed herein is a solid lipid nanoparticle formulation which can increase the water solubility and improve chemical stability of lipophilic compounds such as DHA-dFdC.

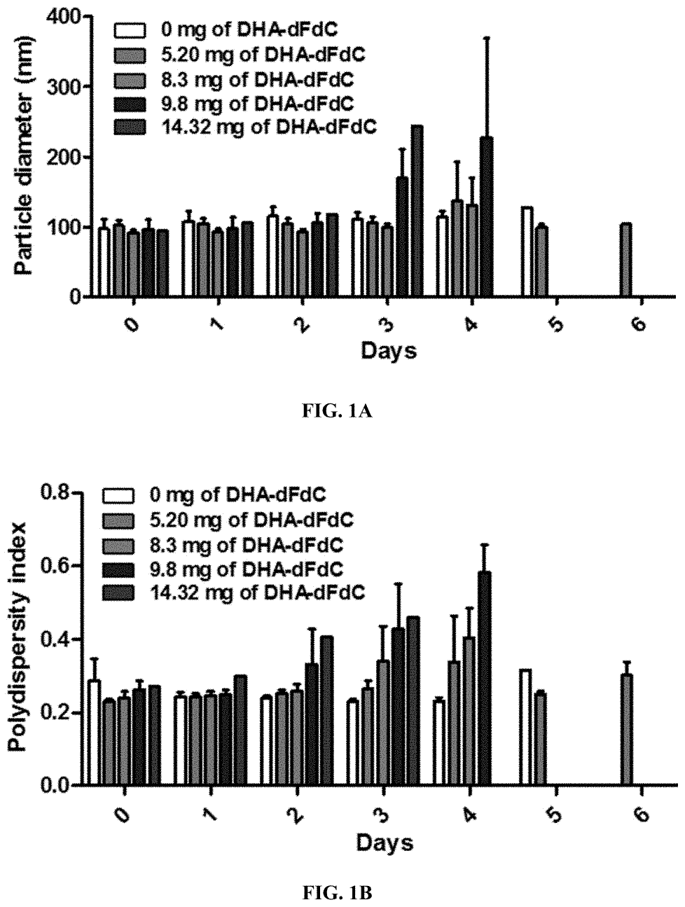

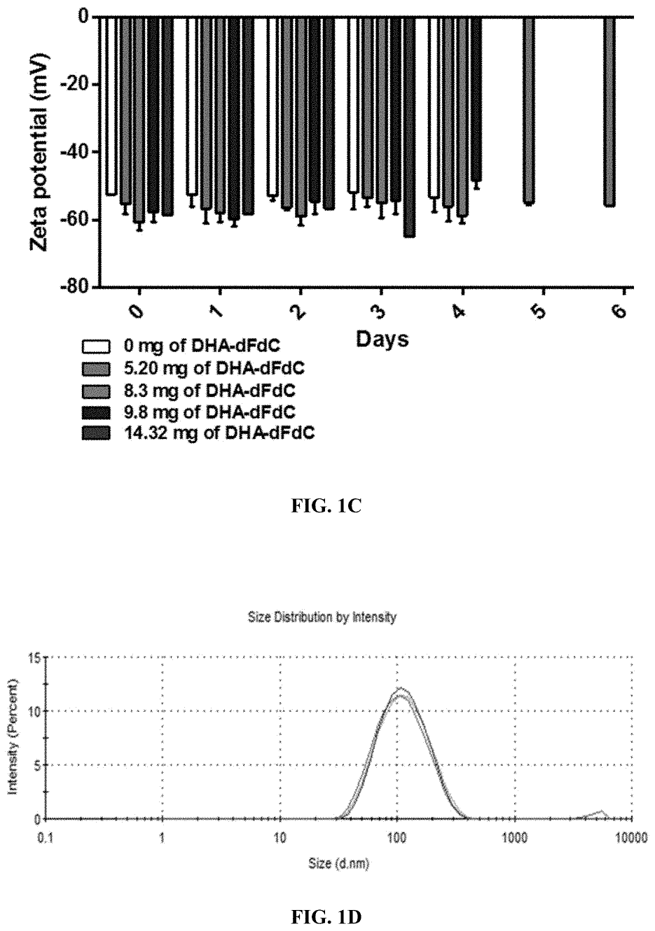

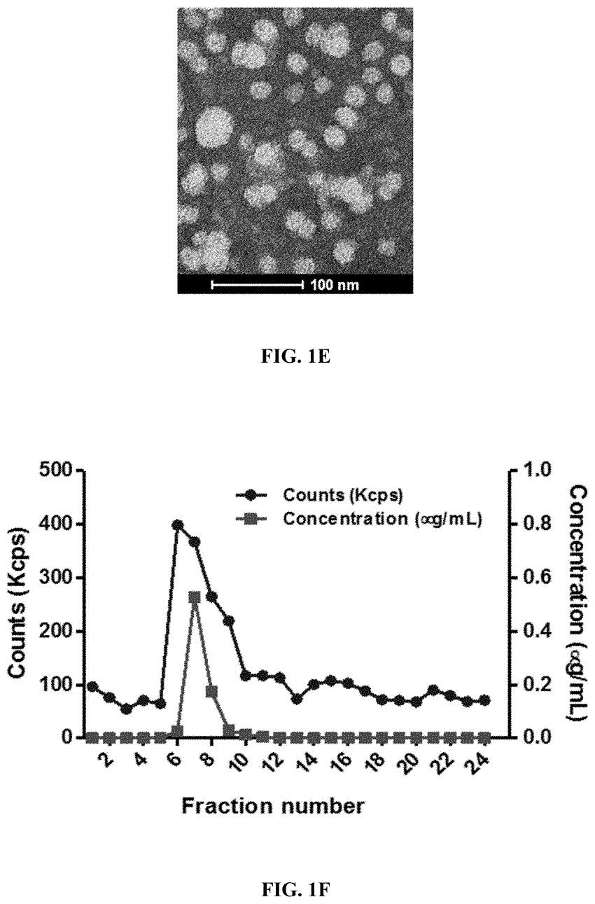

Particle diameter, polydispersity index, and zeta potential of DHA-dFdC-SLNs loaded with various concentrations/amounts of DHA-dFdC are shown in Table 1. Statistical analysis did not reveal any significant differences on the particle sizes and zeta potentials of SLNs prepared with various amounts of DHA-dFdC. However, in a short stability study at 4° C., the DHA-dFdC-SLNs prepared with lower amounts (e.g., 5.2 mg) of DHA-dFdC remained stable after 6 days (FIG. 1A through 1C) and were thus selected for further studies. The example SLN formulation increases the apparent aqueous solubility of DHA-dFdC to at least 5.2 mg/ml. Additional methods to further increase the soluble amount of DHA-dFdC include concentrating the nanoparticles. Shown in FIG. 1D is the dynamic light scattering spectrum of DHA-dFdC-SLNs prepared with 5.2 mg of DHA-dFdC. The TEM images of the DHA-dFdC-SLNs showed that they were spherical (FIG. 1E) with particle size smaller than that determined by dynamic light scattering (FIG. 1D). The encapsulation efficiency of DHA-dFdC in the DHA-dFdC-SLNs was close to 100%, as DHA-dFdC was not detected in the filtrate after ultrafiltration. To corroborate this result, the suspension remained in the ultrafiltration centrifuge tube was re-dissolved in water to extract the DHA-dFdC, and 97%±21.4 (n=6) of DHA-dFC was recovered. There are reports that TPGS as an emulsifier in paclitaxel-loaded polymeric nanoparticles helped to improve paclitaxel encapsulation efficiency to 100% (Zhang et al., Biomat., 2012; 33(19):4889-4906; Mu et al., J. Controlled Release, 2002; 80(1):129-144; Mu et al., J. Controlled Release, 2003; 86(1):33-48). Due to the presence of Tween 20 and TPGS in the disclosed example DHA-dFdC-SLN formulation, it was possible that a certain fraction of the DHA-dFdC was present in micelles. For instance, TPGS has a relative low critical micelle concentration of 0.02% (w/w) at 37° C., ˜1% (w/v) for Tween 20 at 20° C. (Wu et al., Pharma. Tech., 1999, 23(10):52-68; Kim et al., Colloids and Surfaces A: Physicochemical and Engineering aspects, 2001; 187:385-397). Gel permeation chromatography (GPC) was used to identify the extent to which DHA-dFdC was potentially incorporated into micelles (Sloat et al., Intl. J. Pharma., 2011; 409(1):278-288). However, only one apparent DHA-dFdC peak was identified in the GPC spectrum (FIG. 1F), which overlapped with the particle count spectrum, providing additional evidence that almost all the DHA-dFdC was encapsulated into DHA-dFdC-SLNs.

TABLE 1
Characterization of DHA-dFdC-SLNs prepared
with varying amounts of DHA-dFdC.

| | DHA-dFdC (mg) | | | |
|---|---|---|---|---|
| | 0 | 5.2 | 8.3 | 9.8 |
| Particle diameter (nm) | 97.3 ± 13.6 | 102.2 ± 7.3 | 92.0 ± 3.6 | 96.5 ± 14.2 |
| Polydispersity index | 0.27 ± 0.10 | 0.23 ± 0.01 | 0.24 ± 0.02 | 0.26 ± 0.02 |
| Zeta potential (mV) | −51.5 ± 0.1 | −55.3 ± 3.0 | −60.7 ± 2.4 | −57.7 ± 2.9 |

Data shown are mean ± S.D. (n = 3).

Chemical Stability of DHA-dFdC in DHA-dFdC-SLNs after Lyophilization.

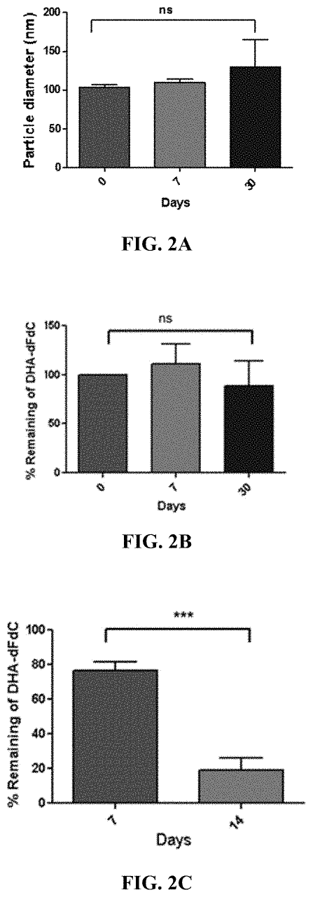

To select a lyoprotectant, various sugars were screened including sucrose, mannitol, and trehalose with concentrations ranging from 2.5% (w/v) to 5% (w/v). Sucrose at concentrations between 2.5% to 3% could effectively prevent particle size change after the DHA-dFdC-SLNs were subjected to lyophilization and reconstitution. Sucrose at 3% (w/v) was thus used as the lyoprotectant for further studies. Particle size of DHA-dFdC-SLNs did not significantly change after 30 days of storage as a lyophilized powder at room temperature (FIG. 2A). Importantly, the content of DHA-dFdC in the lyophilized DHA-dFdC-SLNs powder remained unchanged during the 30 days of storage (FIG. 2B). As a comparison, only 19.1%±7.0 of DHA-dFdC in the DHA-dFdC-vitamin E waxy solid mixture was left after 14 days of storage in the same condition (p<0.0001) (FIG. 2C).

DHA-dFdC in a Tween 80-ethanol-water solution was unstable in storage at room temperature, with a half-life of −14 h (Naguib et al., Neoplasia, 2016; 18(1):33-48). The improved chemical stability of DHA-dFdC in the DHA-dFdC-SLNs dry powder may be attributed to the following three reasons. First, the SLNs may have protected DHA-dFdC incorporated in them from chemical degradation (Geszke-Moritz et al., Mat. Science Engineering: C., 2016, 68:982-994). For example, it was reported that β-carotene loaded in SLNs have improved stability because β-carotene protected against oxidation (Geszke-Moritz et al., Mat. Science Engineering: C., 2016, 68:982-994; 2. Yi et al., J. Agricultural Food Chem., 2014, 62(5):1096-1104). Second, incorporation of TPGS in the formulation may have provided antioxidant properties since TPGS contains α-tocopherol or vitamin E, and TPGS was reported to have more antioxidant activity than free α-tocopherol (Carini et al., Biochem. Pharma., 1990, 39(10):1597-1601; Anstee et al., J. Hepatology, 2010, 53(3):542-550). Third, the SLNs were lyophilized into a dry powder (Vighi et al., Eu. J. Pharma. Biopharma., 2007; 67(2):320-328; Varshosaz et al., Carbohydrate Polymers. 2012; 88(4):1157-1163; do Vale Morais et al., Intl. J. Pharma., 2016; 503 (1-2):102-114).

In Vitro Characterization of DHA-dFdC-SLNs.

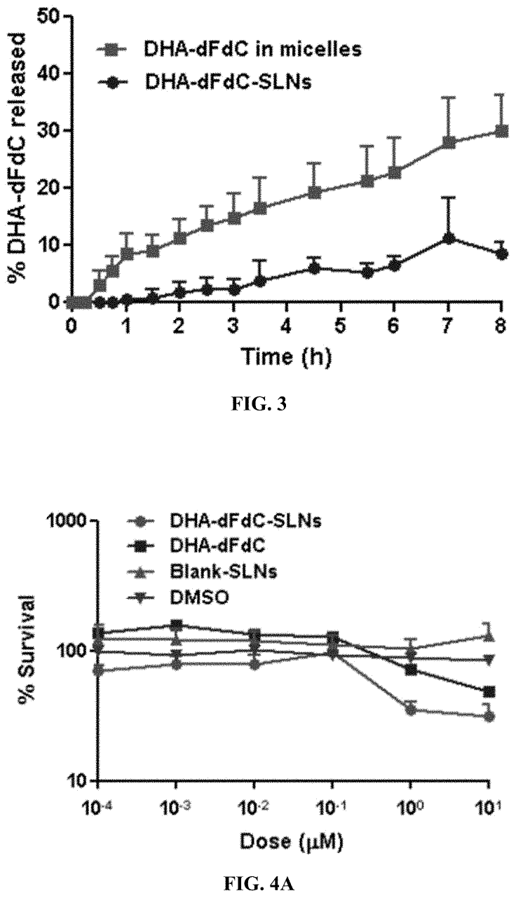

The particles size of DHA-dFdC-SLNs after 18 h of incubation in a simulated biological medium containing 10% FBS in PBS at 37° C. did not increase, suggesting that after intravenous administration, DHA-dFdC-SLNs would not likely aggregate. FIG. 3 shows the release profile of DHA-dFdC from the DHA-dFdC-SLNs. Only 8.6%±1.9 of DHA-dFdC was released from the SLNs within 8 h.

Cytotoxicity of DHA-dFdC-SLNs Against Tumor Cells in Culture.

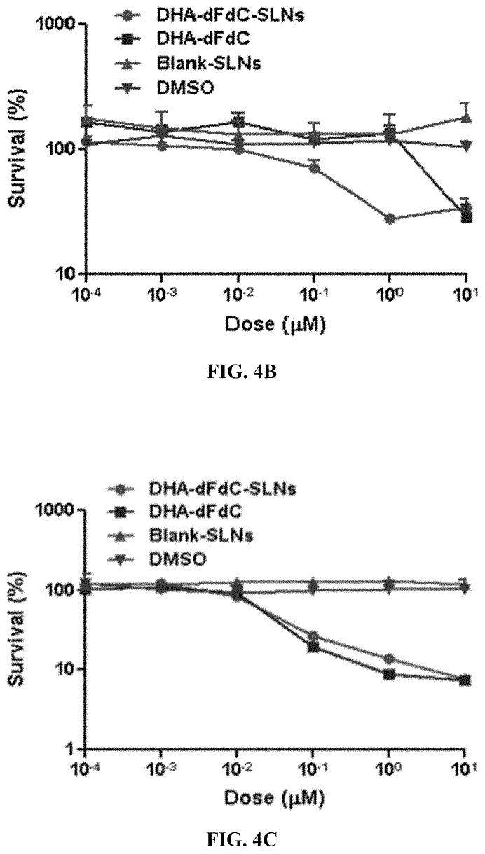

Cytotoxicity of DHA-dFdC-SLNs was evaluated by determining the survival of tumor cells after incubation with SLNs using an MTT assay. DHA-dFdC-SLNs were more cytotoxic than DHA-dFdC in M-Wnt (FIG. 4A; compare IC₅₀ values of 0.92 μM versus 2.15 μM, p<0.05, 24 h of incubation) and B16F10 cells (FIG. 4B; compare IC₅₀ values of 0.085 μM versus 1.81 μM, p<0.0001, 48 h of incubation). In TC-1 cells, the cytotoxicity of DHA-dFdC-SLNs was not significantly different from that of DHA-dFdC (FIG. 4C). Neither DHA-dFdC-free SLNs nor dimethyl sulfoxide (DMSO) vehicle showed significant cytotoxicity in the concentrations tested in all three cell lines (FIGS. 4A-4C).

Plasma Pharmacokinetic of DHA-dFdC in DHA-dFdC-SLNs.

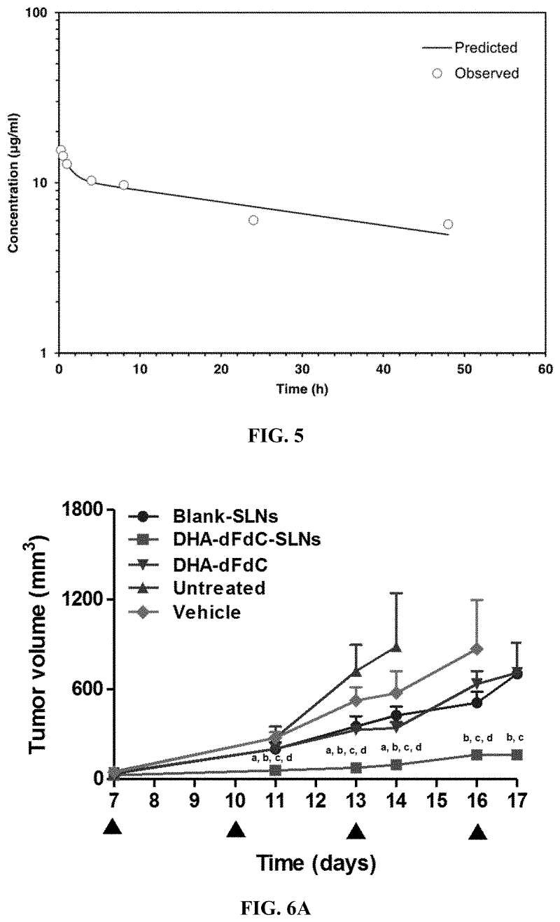

FIG. 5 shows plasma DHA-dFdC levels in mouse plasma samples at different time points after intravenous injection of DHA-dFdC-SLNs. The elimination of DHA-dFdC in mouse plasma followed a bi-exponential model. Table 2 includes selected PK parameters of DHA-dFdC. The AUC_{0-∞} values for DHA-dFdC was 677.3 μg/ml*h, and the plasma half-life of DHA-dFdC in the elimination phase was ˜44 h. By comparison, when DHA-dFdC was given in a Tween 80-ethanol-water solution to mice, the plasma half-life was only ˜58 min (Naguib et al., Neoplasia, 2016; 18(1):33-48).

TABLE 2
Plasma PK parameters of DHA-dFdC-SLNs
when given intravenously to mice.

| Parameter | Unit | Observed |
|---|---|---|
| k₁₀ | 1/h | 0.02 |
| k_{1/2} | 1/h | 0.32 |
| k₂₁ | 1/h | 0.58 |
| t_{1/2α} | h | 0.76 |
| t_{1/2β} | h | 43.95 |
| C₀ | μg/ml | 16.85 |
| V | ml | 0.12 |
| CL | ml/h | 0.03 |
| V₂ | ml | 0.07 |
| cL₂ | ml/h | 0.04 |
| AUC_{0-24 h} | (μg/ml)*h | 362.82 |
| AUC_{0-inf} | (μg/ml)*h | 677.30 |
| AUMC | (μg/ml)*h² | 42519.06 |
| MRT | h | 62.78 |

Antitumor Activity of DHA-dFdC-SLNs in Mice.

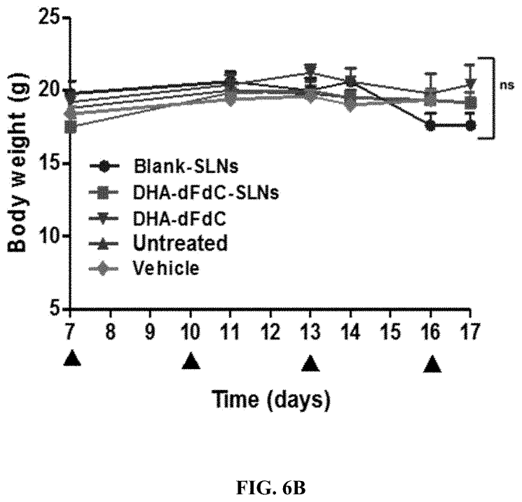

The antitumor activity of DHA-dFdC-SLNs was evaluated in mice with pre-established B16-F10 tumors. Tumors grew aggressively when mice were left untreated or treated with the Tween 80-ethanol-in-water vehicle only (FIG. 6A). DHA-dFdC in solution and Blank-SLNs lacking DHA-dFdC at the tested dosing regimen delayed tumor growth by 4 days, but there were no significant difference between tumor size in mice treated with DHA-dFdC in solution or Blank-SLNs and the sizes of tumors in mice left untreated in all the days compared (FIG. 6A). DHA-dFdC-SLN treatment was the most effective in inhibiting the tumor growth. The DHA-dFdC-SLN nanoparticle formulation delayed tumor growth by about 8 days, and tumor size in DHA-dFdC-SLN-treated mice were significantly smaller than those in untreated mice or mice treated with DHA-dFdC in solution (FIG. 6A). There was no significant difference in body weights of mice among the groups during the treatments (FIG. 6B), indicating DHA-dFdC-SLNs at the dosing regimen tested were well tolerated.

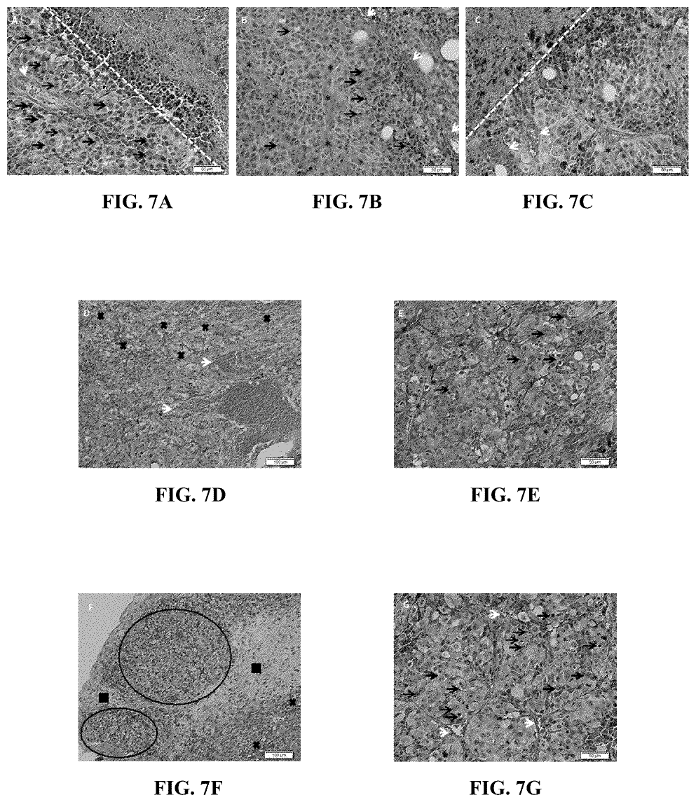

FIG. 7 shows representative H&E images of B16-F10 tumors from mice in different groups. Tumors in mice that were left untreated (FIG. 7A) or treated with vehicle (FIG. 7B) or DHA-dFdC-free SLNs (FIG. 7C) were in a late tumor stage with large blood vessels with large lumen. In addition, tumors in these groups showed large necrotic areas, increased desmoplasia, and vascular collapse (FIGS. 7A-7C). In solid tumors such as melanoma, high interstitial fluid constitutes a significant barrier to chemotherapy as it can induce compression of blood vessels, diverting blood from the center of tumors to the periphery, which reduces the transcapillary transport of chemotherapeutics (Pautu et al., Pharma. Res., 2017). Tumor treated with DHA-dFdC-SLNs showed a higher number of blood vessels with small lumen (FIG. 7G). In addition, an increasing level of connective tissue can be observed around the tumoral zone in tumors in mice treated with DHA-dFdC-SLNs (FIG. 7F). This fibrous connective tissue likely has a tumor encapsulation effect, providing a protective barrier to tumor local and vascular invasion (Ng et al., Cancer, 1992; 70(1):45-49). As an example of the protective effects of tumor encapsulation, patients with liver metastasis have a better prognostic when metastasis encapsulation occurs by the formation of a fibrotic capsule (Morino et al., Clinico-pathological features of liver metastases from colorectal cancer in relation to prognosis. 1991. Ohlsson et al., World J. Surgery, 1998; 22(3):268-277; Lunevicius et al., J. Cancer Res. Clin. Oncology, 2001; 127(3):193-199). Indeed, the formation of capsules protects the liver parenchyma from cancer invasion (Lunevicius et al., J. Cancer Res. Clin. Oncology, 2001; 127(3):193-199). Thus, DHA-dFdC-SLNs can be used to treat melanoma, for instance by inducing protective tumor encapsulation which can aid in avoiding metastasis and facilitate surgical removal.

In contrast, tumors in mice treated with DHA-dFdC alone showed vascular collapse, high desmoplasia, and necrotic areas (FIGS. 7D and 7E). Tumors in mice treated with DHA-dFdC-SLNs showed more cells in apoptosis, but less cells in necrosis, as compared to tumors in mice treated with DHA-dFdC alone in solution or untreated controls. DHA-dFdC-free SLNs (Blank-SLNs) showed a tendency to delay tumor growth as compared to the untreated group (FIG. 6A). In vivo and in vitro studies reported that TPGS had anticancer activity as a single agent, being able to inhibit the growth of human prostate and lung carcinoma cells (Youk, et al., J. Controlled Release, 2005, 107, (1), 43-52; Vighi, et al., Eu. J. Pharma. Biopharma., 2007, 67, (2), 320-328). Furthermore, TPGS can selectively induce apoptosis in T cell acute lymphocytic leukemia (ALL) or Jurkat clone E6-1 cells through the induction of oxidative stress pathway (Ruiz-Moreno, et al., Apoptosis, 2016, 21, (9), 1019-1032). In addition, TPGS was reported to selectively induce cell cycle arrest and apoptosis in breast cancer cell lines such as MCF7 and MDA-MB-231, but not in “normal” immortalized cells such as MCF-10A and MCF-12F (Neophytou, et al., Biochem. Pharma., 2014, 89, (1), 31-42). Finally, a synergistic effect between TPGS₂ₖ and docetaxel was reported in MCF-7 cell lines, wherein the incubation of MCF-7 cells with TPGS₂ₖ micelles without docetaxel induced cytotoxicity (Mi, et al., Biomat., 2011, 32, (16), 4058-4066). One reason that could explain the lack of cytotoxicity by DHA-dFdC-free SLNs in culture cells is the low concentration of TPGS used in the formulation 1.5 mM). Indeed, higher concentrations TPGS were used in culture to induce cell cytotoxicity (e.g. >10 mM), and in animal studies to suppress tumor growth 40 mM) (Youk, et al., J. Controlled Release, 2005, 107, (1), 43-52; Constantinou, et al., Nutrition Cancer, 2012, 64, (1), 136-152; Ruiz-Moreno, et al., Apoptosis, 2016, 21, (9), 1019-1032; Neophytou, et al., Biochem. Pharma., 2014, 89, (1), 31-42).

In summary, disclosed herein is a solid lipid nanoparticle comprising DHA-dFdC in which all materials used in the formulation are biocompatible. Indeed, lecithin, GMS, and Tween 20 are GRAS materials for parenteral administration (Rowe, et al., Pharmaceutical Press, 6th ed.; 2009). TPGS has been approved by the FDA as a safe pharmaceutical adjuvant that allows its use parenteral pharmaceutical formulations. Id. Moreover, the method of preparing the SLN formulation is straight forward and scalable for industrial manufacturing. In addition, the small size of the DHA-dFdC-SLNs (102.2±7.3 nm) facilitates sterilization by filtration (0.2 μm). Finally, toxic organic solvents were not used when preparing the SLNs during the emulsion preparation, thereby avoiding an evaporation process and residual solvent in the formulation. As to the mechanism underlying the improved antitumor activity of the DHA-dFdC-SLNs in the animal model tested, the enhance permeability and retention effect (EPR) was likely responsible (Bazak, et al., Mol. Clin. Oncol., 2014, 2, (6), 904-908).

### Example 2. Oral SLN Formulation Having Improved DHA-dFdC Bioavailability

Certain nanocarriers (e.g. SLNs, liposomes, nanoemulsions, micelles, and polymeric nanoparticles) have gained some attention for improving oral delivery of anticancer drugs by increasing the apparent solubility of drugs, reducing degradation of drugs within the GI tract, and/or improving drug absorption (Date, et al., J. Controlled Release, 2016, 240, 504-526; Thanki, et al., J. Controlled Release 2013, 170, (1), 15-40; Lin, et al., J. Food Drug Analysis, 2017, 25, (2), 219-234). This example discloses that DHA-dFdC-SLNs surprisingly can enable oral administration of DHA-dFdC, a highly lipophilic compound.

### Materials and Methods

Materials and cell lines. Mannitol, Tween 20, GMS, TPGS, sodium chloride (NaCl), hydrochloric acid (HCl, 37%), monobasic potassium phosphate (KH₂PO₄), sodium hydroxide (NaOH), and Tween 80 were from Sigma-Aldrich (St. Louis, Mo.). Gemcitabine HCl was from Biotang, Inc. (Lexington, Mass.). Soy lecithin was from Alfa Aesar (Ward Hill, Mass.). Ethyl acetate (EtOAc), tetrahydrofuran (HPLC-grade), isopropanol, and methanol (HPLC-grade) were from Thermo Fisher Scientific (Waltham, Mass.). Float-A-Lyzer®G2 dialysis device (MWC 50 kD) was from Spectrum Inc. (New Brunswick, N.J.).

Murine melanoma (B16-F10) cancer cell lines were from the American Type Culture Collection (Manassas, Va.). B16-F10 cells were grown in DMEM (Invitrogen, Carlsbad, Calif.) supplemented with 10% (v/v) fetal bovine serum (FBS), 100 U/mL of penicillin, and 100 μg/mL of streptomycin, all from Invitrogen (Carlsbad, Calif.).

### Synthesis of 4-(N)-docosahexaenoyl 2′,2′-difluorodeoxycytidine (DHA-dFdC

DHA-dFdC was synthesized as published (Naguib, et al., Neoplasia, 2016, 18, (1), 33-48). The purity of the resultant DHA-dFdC was confirmed by NMR and Mass Spectrum analyses.

Preparation and characterization of 4-(N)-docosahexaenoyl 2, 2′-difluorodeoxycytidine nanoparticles (DHA-dFdC-SLNs). Solid Lipid Nanoparticles (SLNs) were prepared as described in Example 1.

DHA-dFdC was extracted from the nanoparticles to determine concentration. Briefly, 100 μL of DHA-dFdC-SLNs were mixed with 100 μL of isopropanol, vortexed for 30 s, and maintained at room temperature. Five minutes later, 600 μL of ethyl acetate was added. The mixture was vortexed per 30 s and centrifuged at 11,000 rcf for 20 min. The supernatant was collected into a glass vial. After solvent was evaporated under nitrogen, the sample was re-dissolved in 100 μL THF, and concentration of DHA-dFdC was measured by HPLC (Naguib, et al., Neoplasia, 2016, 18, (1), 33-48).

Stability of DHA-dFdC-SLNs in Stimulated Gastrointestinal Fluids.

Stability of DHA-dFdC-SLNs in simulated gastric fluid (SGF, pH 1.2) and simulated intestine fluid (SIF, pH 6.8) without enzymes was evaluated. SGF and SIF were prepared according USP XXVI. The SGF was prepared by dissolving 2 g of NaCl into 7 mL of HCl, and completed the volume to 1000 mL with deionized water (Wang, et al., Oncotarget, 2017, 8, (52), 89876). SIF was prepared by adding 6.8 g of KH₂PO₄ and 896 mg NaOH into 1000 mL of deionized water. Id. DHA-dFdC-SLNs were incubated in SGF or SIF media at 37° C. under agitation (100 rpm). At different time points (e.g., 0, 1, 2, 4, and 6 h), samples were taken and diluted into water to measure particle size using Malvern Zetasizer Nano ZS. As a control, DHA-dFdC-SLNs were incubated in phosphate-buffered saline (PBS, 10 mM, pH 7.4).

Transmission Electron Micrographs (TEM).

Size and morphology of DHA-dFdC-SLNs before and after incubation in SGF and SIF were examined using a transmission electron microscope as described in Example 1.

In Vitro Release in Simulated Gastrointestinal Fluids.

To test the release behavior of DHA-dFdC from DHA-dFdC-SLNs in SGF and SIF, DHA-dFdC-SLNs in SGF or SIF were placed into a 1 mL of cellulose ester dialysis tube (151 μg/mL of DHA-dFdC), which was then placed in a plastic conical tube containing 13 mL of dissolution media (SGF or SIF with 2.5% of Tween 20) to create a sink condition. The plastic tube was placed in a thermostatic shaker at 37° C. at 100 rpm (Max Q 200, Thermo Fisher Scientific). At predetermined time points, 200 μL of the release medium was withdrawn and subsequently replaced with an equal volume of fresh medium. The concentration of DHA-dFdC in the medium was determined using HPLC. As a control, 151 μg of DHA-dFdC was dissolved in 2.5% of Tween 20 to confirm that the diffusion of DHA-dFdC across the dialysis tube membrane was not rate-limiting.

Pharmacokinetic Studies.

The Institutional Animal Care and Use Committee at The University of Texas at Austin approved the animal protocol. Female C57BL/6 mice (6-8 weeks, Charles River Laboratories, Wilmington, Mass.) were fasted for 3 h. Water was allowed ad libitum. Mice were orally gavaged with DHA-dFdC dissolved in a vehicle solution (Tween 80 (10%, w/v), ethanol (5.2% v/v), and mannitol (5%, w/v) in sterile water) (Naguib, et al., Neoplasia, 2016, 18, (1), 33-48; Valdes, et al., Pharma. Res., 2017, 34, (6), 1224-1232) or the DHA-dFdC-SLNs suspended in a sterile mannitol solution (5%, w/v), or intravenously injected with the DHA-dFdC-SLNs suspended in a sterile mannitol solution (5%, w/v). The dose of DHA-dFdC was 2 mg per mouse. Mice (n=3) were euthanized at various time points (e.g., 0.25, 0.5, 1, 2, 5, 8, 12, and 24 h). Blood was collected into heparin-coated tubes, which were then centrifuged at 13,000 rcf for 20 min to isolate plasma. The plasma (200 μL) was mixed with 200 μL of isopropanol and 200 μL of cold PBS, vortexed and then incubated at 4° C. for 5 min Following incubation, 1000 μl of ethyl acetate was added. The mixture was vortexed for 5 min, followed by centrifugation at 18,000 rcf for 5 min. The supernatant was collected and dried under nitrogen. Finally, the residue was re-dissolved in 100 μl of THF, which was then analyzed using HPLC (Naguib, et al., Neoplasia, 2016, 18, (1), 33-48). As internal control 4-(N)-stearoyl 2′,2′-difluorodeoxycytidine (GemC18) was added in the samples before extraction (Sloat, et al., Intl. J. Pharma., 2011, 409, (1), 278-288). Data were analyzed using PK Solver®, assuming a two-compartmental model (Wang, et al., Oncotarget, 2017, 8, (52), 89876).

Antitumor Activity of Orally Administered DHA-dFdC-SLNs in a Tumor-Bearing Mouse Model.

Female C57BL/6 mice (18-20 g) were subcutaneously (s.c) injected with B16-F10 (5×10⁵ cells/mouse) in the right flank on day 0. Seven days later, mice were randomized into 4 groups (n=7-8) and orally gavaged with DHA-dFdC (250 μg/mouse) dissolved in vehicle (Naguib, et al., Neoplasia, 2016, 18, (1), 33-48; Valdes, et al., Pharma. Res., 2017, 34, (6), 1224-1232), DHA-dFdC-SLNs (250 μg/mouse of DHA) dispersed in mannitol 5%, or DHA-dFdC-free SLNs dispersed in mannitol 5%. As a control, one group of mice were left untreated. Treatment was repeated every day until day 11. Mice were allowed to rest for two days, and treatment was resumed on day 13 and continued until day 20. Mice were monitored daily until the endpoint (e.g., death, tumor size reaching 15 mm, tumor ulceration, body weight loss of more than 20%, or other signs of severe distress and discomfort).

Statistical analysis.

Statistical analyses were completed by one-way ANOVA followed by a Bonferroni post hoc test. Mouse survival curves were compared using the Mantel-Cox log-rank method. A p value of <0.05 (two-tail) was considered significant. Most of the analyses were performed with GraphPad Prism (GraphPad Software, Inc., La Jolla, Calif.). Pharmacokinetic parameters were obtained using PK Solver® (Zhang, et al., Comp. Meth. Prog. Biomed., 2010, 99, (3), 306-314).

### Results and Discussion

The use of solid-lipid nanoparticles for oral drug administration provides several advantages, such as improving the stability, enhancing the bioavailability of the drug and decreasing its toxicity (Lin, et al., J. Food Drug Analysis, 2017, 25, (2), 219-234; Uner, et al., Intl. J. Pharma. Sci., 2005, 60, (8), 577-582; Lim, et al., J. Controlled Release, 2004, 100, (1), 53-61; Yuan, Intl. J. Nanomed., 2014, 9, 4829; MuEller, et al., Euro. J. Pharma. Biopharma., 2000, 50, (1), 161-177). DHA-dFdC-SLNs by incorporating DHA-dFdC into solid lipid nanoparticles prepared with soy lecithin, GMS, TPGS, and Tween 20 to overcome the poor water solubility and chemical instability of DHA-dFdC, as described in Example 1. The main characteristics of the DHA-dFdC-SLNs are summarized in Table 3. The diameter of the nanoparticles is 101±8 nm. Particle size (diameter) significantly affects gastrointestinal absorption, and nanoparticles with a particle diameter lower than 300 nm are good candidate for oral administration (Thanki, et al., J. Controlled Release 2013, 170, (1), 15-40). Indeed, an evaluation of the cellular uptake of polymeric nanoparticles such as Vitamin E TPGS-coated PLGA nanoparticles or PVA-coated PLGA nanoparticles by Caco-2 cells in culture showed that the most desirable particles size is in the range of 100-200 nm (Win, et al., Biomat., 2005, 26, (15), 2713-2722). The zeta potential of DHA-dFdC-SLNs was −44±2 mV, indicating their stability in an aqueous suspension (Win, et al., Biomat., 2005, 26, (15), 2713-2722; Aditya, et al., J. Agri. Food Chem., 2013, 61, (8), 1878-1883).

TABLE 3
Characterization of DHA-dFdC-SLNs
used for gastrointestinal studies.

| | |
|---|---|
| DHA-dFdC (mg) | 5.2 |
| Particle diameter (nm) | 100.5 ± 7.7 |
| Polydispersity index | 0.214 ± 0.030 |
| Zeta potential (mV) | −43.5 ± 2.2 |
| Entrapment efficiency % | 97.0% ± 21.4 |

Data shown are mean ± S.D. (n = 3)

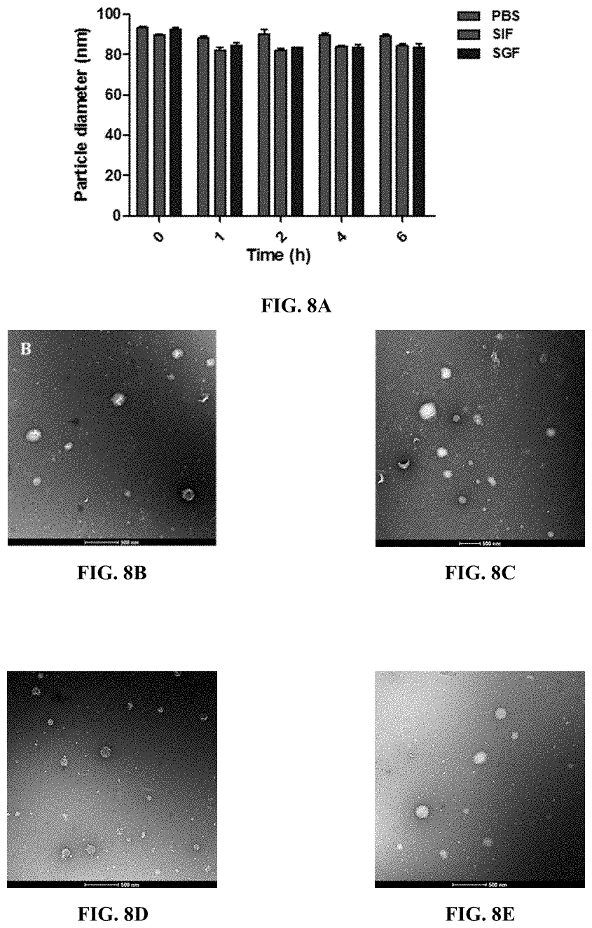

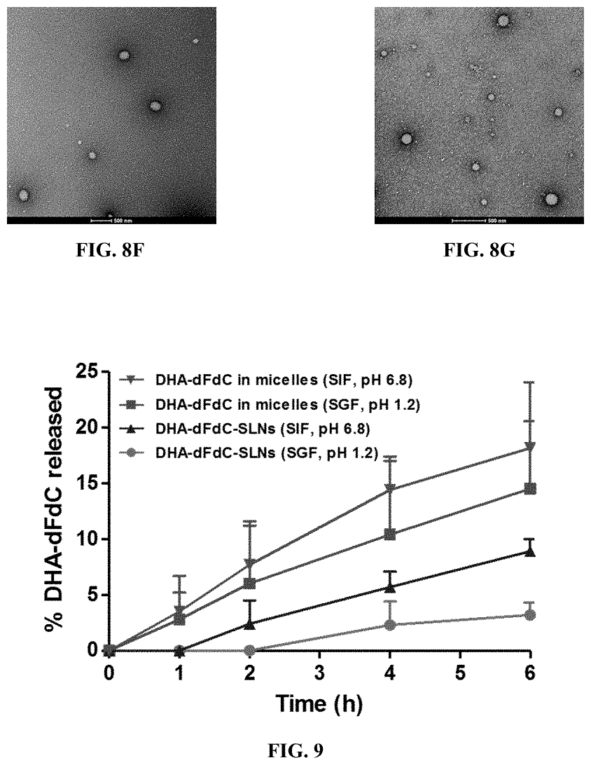

Stability of DHA-dFdC-SLNs in stimulated gastrointestinal fluids. In vitro stability of DHA-dFdC-SLNs in simulated gastrointestinal (GI) fluid (e.g. SGF or SIF) was examined. As a control, stability of DHA-dFdC-SLNs in PBS (10 mM, pH 7.4) was also included. Particle diameter of DHA-dFdC-SLNs as measured by DLS did not increased during 6 hours (h) of incubation in SGF or SIF (FIG. 8A). Indeed, particle size decreased slightly (˜5.4% in SIF and 6.1% in SGF, as compared to in PBS) (FIG. 8A). Shown in FIG. 8B-8G are representative TEM images of the nanoparticles before and after 6 h of incubation in SGF or SIF. Overall, nanoparticle shape did not change significantly after incubation; however, after 6 h of incubation in SIF, the surface of the DHA-dFdC-SLNs appeared rough (FIG. 8E, inset). This rough appearance was not observed after DHA-dFdC-SLNs were incubated in the SGF (FIG. 8G, inset). Studies examining the degradation of SLNs in GI fluids showed that their degradation induces a decrease in particle size due to the loss of surfactant coated on the nanoparticle surface, ultimately leading to an increase in particle diameter due to aggregation in the absence of surfactant (Aditya, et al., J. Agri. Food Chem., 2013, 61, (8), 1878-1883; Muller, et al., Intl. J. Pharma., 1996, 144, (1), 115-121). Non-ionic surfactants such as Tween 80, Tween 20, Tween 60, and PVA provide steric stabilization to particles in acid pH (Van Aken, et al., Food Hydrocolloids, 2011, 25, (4), 781-788). Tween 20 was used as a surfactant in DHA-dFdC-SLNs, which might explain the stability of these nanoparticles in SGF. TPGS is a non-ionic surfactant as well, and the presence of TPGS in DHA-dFdC-SLNs may have also contributed to the stability of the nanoparticles in simulated GI fluids.

In Vitro Release in Simulated Gastrointestinal Fluids.

The in vitro release profiles of DHA-dFdC from DHA-dFdC-SLNs in simulated GI fluids is shown in FIG. 9. After 6 h, the cumulative release of DHA-dFdC reached—8.9% and ˜3.2% in SIF and SIG, respectively. Release of DHA-dFdC from the DHA-dFdC-SLNs was monitored for 6 h only, because the GI transition time in mice is 6-8 h (Zhao, et al., J. Pharma. Sci., 2010, 99, (8), 3552-3560). As shown in the insert of FIG. 8E, the surface of SLNs was not smooth after 6 h of incubation in SIF, indicting erosion of the particles, which may explain the faster release of DHA-dFdC from SLNs in SIF.

Oral Bioavailability of DHA-dFdC in DHA-dFdC-SLNs.

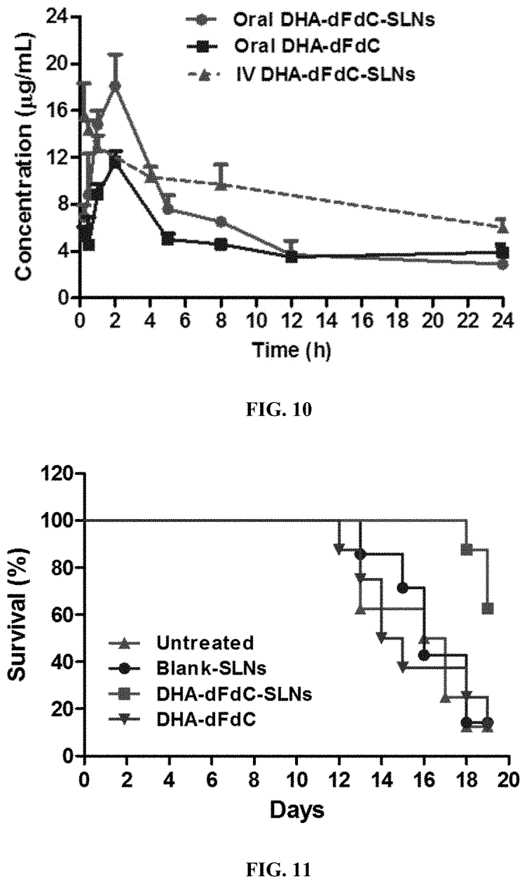

Plasma concentrations of DHA-dFdC at different time points after oral administration or intravenous injection of the DHA-dFdC-SLNs in suspension at 2 mg of DHA-dFdC per mouse are shown in FIG. 10. Selected pharmacokinetic parameters of DHA-dFdC are summarized in Table 4.

TABLE 4
Selected pharmacokinetics parameters of DHA-dFdC in
plasma followed by i.v. administration of DHA-dFdC-
SLNs or oral administration of DHA-dFdC in Tween 80/ethanol/water
solution or in DHA-dFdC-SLNs.

| | Oral | | i.v. |
|---|---|---|---|
| | administration | | administration |
| PK | DHA-dFdC- | DHA- | DHA-dFdC- |
| parameters | SLNs | dFdC | SLNs |
| Dose (mg) | 2 | 2 | 2 |
| k₁₂ (1/h) | 0.41 | 0.56 | 0.40 |
| T_{1/2α} (h) | 1.10 | 1.07 | 0.53 |
| T_{1/2β} (h) | 32.76 | 693147.18 | 25.58 |
| Tₘₐₓ (h) | 1.73 | 1.75 | — |
| Cₘₐₓ (μg/mL) | 17.01 | 10.50 | — |
| AUC 0-24 μg*h/mL) | 143.44 | 113.55 | 210.58 |
| Fab % | 68.12 | — | — |
| Frel % | 126.32 | — | — |

AUC: total area under the plasma concentration-time curve form time zero to 24 h; Cₘₐₓ: peak plasma concentration; Tₘₐₓ: time to reach Cₘₐₓ; Frel %: relative oral bioavailability in percentage; Fab %: absolute oral bioavailability in percentage.

Plasma DHA-dFdC level after i.v. administration of DHF-dFdC-SLNs in healthy mice followed a two-compartment model with AUC_{0-24 h} value of 210.58 μg*h/mL. On the other hand, the plasma DHA-dFdC level in mice after oral administration of DHA-dFdC-SLNs followed an apparent adsorption phase and then a clearance phase, with a C. of 17.01 μg/mL, T. of 1.73 h, and AUC_{0-24 h} of 143.44 μg*h/mL. The absolute oral bioavailability of DHA-dFdC in the DHA-dFdC-SLNs was 68.12% based on the AUC₀₋₂₄ h values in Table 4.

In comparison, the plasma concentration of DHA-dFdC-time curve of the DHA-dFdC after it was orally administered in a Tween 80-ethanol-water solution is shown in FIG. 10. The T. was—1.7 h, similar to that of oral DHA-dFdC in SLNs (Table 2). However, the C. and AUC_{0-24 h} values of the DHA-dFdC in solution were found to be 10.50 μg/mL and 113.55 μg*h/mL, respectively. Therefore, the bioavailability of DHA-dFdC in the DHA-dFdC-SLNs, relative to that in the Tween 80-ethanol in water solution, was 126.4%.

The exact mechanism by which the DHA-dFC in the DHA-dFdC-SLNs was absorbed into the blood circulation after oral gavage is unknown. Generally, orally administered SLNs can be absorbed as intact particles through the microfold cells in the Peyer's patches and then transported to the lymphatic system (Li, et al., J. Controlled Release, 2009, 133, (3), 238-244). However, others have suggested that SLNs suffer from digestion or degradation in the GI tract, and only a very small fraction, if any, of orally administered SLNs can reach the blood circulation intact (Hu, et al., Nanoscale, 2016, 8, (13), 7024-7035). Of course, DHA-dFdC can be released from the SLNs in the GI tract (as shown in vitro in FIG. 9), especially in the presence of lipases and co-lipases from pancreas. DHA-dFdC could then be absorbed by passive diffusion or with the help of biles in the GI tract (Thomson, et al., Canad. J. Phys. Pharma., 1989, 67, (3), 179-191; Porter, et al., Nat. Rev. Drug Disc., 2007, 6, (3), 231).

As to the higher bioavailability of DHA-dFdC in SLNs relative to DHA-dFdC in Tween 80/ethanol/water solution, the DHA-dFdC in the solution may be susceptible to precipitation when orally administered, which can lead to a decrease in bioavailability (Naguib, et al., Neoplasia, 2016, 18, (1), 33-48). Higher levels of exogenous lipids from SLNs after digestion (e.g., by exogenous solubilizing components), relative to endogenous solubilizing components in the GI tract, may lead to a change in the nature of the GI fluid and enhance DHA-dFdC solubilization (Porter, et al., Nat. Rev. Drug Disc., 2007, 6, (3), 231). Nonetheless, DHA-dFdC in solution contained Tween 80, which may explain the relatively high oral bioavailability of DHA-dFdC in the tested solution (Seeballuck, et al., Pharma. Res., 2004, 21, (12), 2320-2326). Tween 80 can be digested by intestinal cells to release oleic acid, which can be used to increase basolateral secretion of triglyceride-rich lipoproteins such as chylomicrons, increasing the lymphatic uptake of lipophilic drug. Id. In addition, Tween 80 can inhibit intestinal P-gp efflux, increasing the concentration and residence time into the enterocyte of P-gp substrate (Nerurkar, et al., Pharmal. Res., 1996, 13, (4), 528-534). Although Tween 80 can inhibit intestinal P-gp activity, it is less effective compared to TPGS (Guo, et al., Euro. J. Pharma. Sci., 2013, 49, (2), 175-186). TGPS as an emulsifier in a paclitaxel-polymeric nanoparticle formulation helped to increase the oral bioavailability of paclitaxel by 10-fold, as compared to oral Taxol (Zhao, et al., J. Pharma. Sci., 2010, 99, (8), 3552-3560). Furthermore, TPGS1000-emulsified SLNs improved the intestinal absorption and relative oral bioavailability of docetaxel in rats (Cho, et al., Intl. J. Nanomed., 2014, 9, 495). Of course, it is unknown whether DHA-dFdC is a substrate of P-gp. Therefore, the high oral bioavailability of DHA-dFdC in DHA-dFdC-SLNs may be attributed in part to the presence of TPGS in the formulation as well.

Antitumor Activity of DHA-dFdC-SLNs in a Tumor-Bearing Mouse Model.

DHA-dFdC-SLNs antitumor activity was evaluated in a mouse melanoma model. In Example 1, it was shown that DHA-dFdC-SLNs significantly inhibited growth of B16-F10 tumor cells in culture and in mice when given intravenously. Consequently, B16-F10 tumor-bearing mice were used to test DHA-dFdC-SLN antitumor activity when given orally.

DHA-dFdC-SLNs were orally gavaged at a dose of 250 μg of DHA-dFdC per mouse daily for a total of 12 days (with a two-day rest in the middle). Fifty percent (50%) of mice in the untreated group reached the endpoint on day 16 (FIG. 11). Oral DHA-dFdC-SLNs significantly improved the survival, as compared to the untreated group (p<0.05). Oral DHA-dFdC in Tween 80/ethanol/water solution did not significantly affect mouse survival as compared to untreated mice, which was surprising because the bioavailability of the DHA-dFdC in the Tween 80/ethanol/water solution was—54% (Table 2). Toxicity associated with repeated dosing of the DHA-dFdC in Tween 80/ethanol/water solution was likely related to the lack of survival advantage of the DHA-dFdC solution over untreated mice, as 62.5% of the mice orally gavaged with the DHA-dFdC in Tween 80/ethanol/water solution showed signs of toxicity such as a body weight decrease of more than 20% (one mouse) or severe tumor ulceration (four mice). The exact reasons underlying the toxicity of the DHA-dFdC in the Tween 80/ethanol-water solution remains unknown, but could be related to the Tween 80-ethanol-water solution, although the amounts of Tween 80 and ethanol taken by mice from the DHA-dFdC in Tween 80/ethanol/water solution were within the normal range recommended for preclinical animal study (e.g., water containing a maximum of 10% Tween 80 and 5% ethanol is well tolerated) (Gad, et al., Intl J. Toxicol., 2006, 25, (6), 499-521; Shimizu, et al., Labor. Mouse, 2004, 527-541). Nonetheless, the mouse survival data clearly indicate the SLN formulation reduced the oral toxicity of DHA-dFdC as well.

### Example 3. Additional SLN Formulations

### DHA-dFdC and Varying Concentrations of TPGS

DHA-dFdC (5 mg), 3.5 mg soy lecithin, 0.5 mg glycerol monostearate, and TPGS at different amounts (0.4375, 0.875, or 1.75 mg) were mixed and dispersed in 800 μl of de-ionized and filtered (0.22 μm) hot water (80° C.). The mixture was vortexed, sonicated for 10 minutes, and then maintained on an 80° C. hot plate while stirring at 800 rpm for 5 minutes. Separately, 55 mg of Tween 20 was dissolved in 1 ml of hot water, and then 200 ml of this solution were added dropwise into the mixture to reach a final concentration of 1% (v/v) Tween 20. The emulsions were cooled to room temperature while stirring to form nanoparticles.

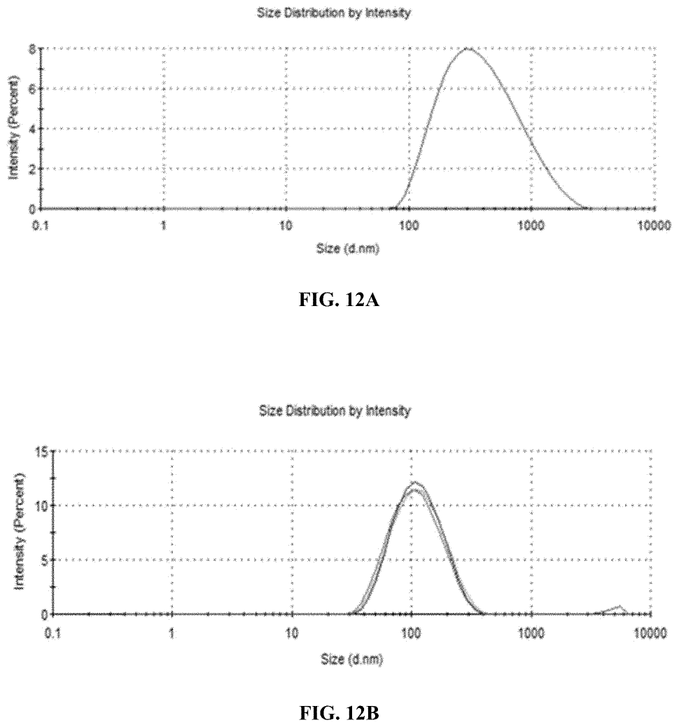

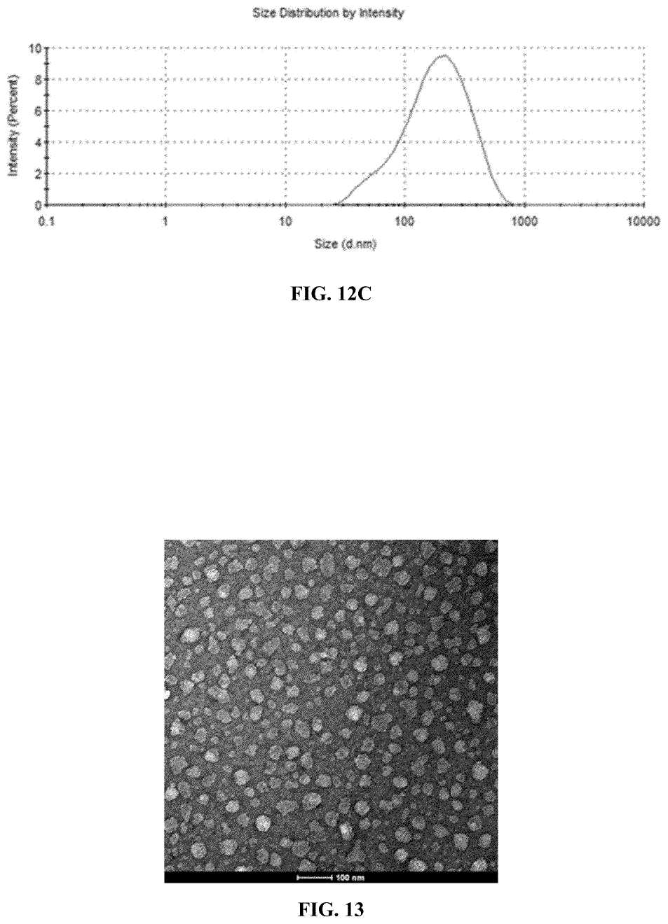

Particle diameter, polydispersity index (PDI), and zeta potential of the nanoparticles were determined using a Malvern Zeta Sizer Nano ZS (Westborough, Mass.). Results are summarized in FIGS. 12A-12C and Table 5. Nanoparticles prepared with 0.4375 mg TPGS were undesirably large (more than 50% of particles were above 400 nm, FIG. 12A), while those prepared with 0.875 mg TPGS had desirable particle diameter, size distribution, and polydispersity index (FIG. 12B, Table 5).

TABLE 5
Characterization of DHA-dFdC-SLNs with 0.875 mg of TPGS.

| TPGS (mg) | Particle diameter (nm) | PDI | Zeta potential (mV) |
|---|---|---|---|
| 0.875 | 102.2 ± 7.3 | 0.23 ± 0.01 | −55.3 ± 3.0 |

### DHA-Lecithin-GSM-TPGS

Docosahexaenoic acid (DHA) (5.5 mg), 3.5 mg soy lecithin, 0.5 mg glycerol monostearate, and 1.75 mg vitamin E-TPGS (TPGS) were mixed in 800 μl of de-ionized and filtered (0.22 μm) hot water (80° C.). The mixture was vortexed, sonicated for 10 minutes, then maintained on a 80° C. hot plate while stirring at 800 rpm for 5 minutes. The emulsions were cooled to room temperature while stirring to form nanoparticles. Finally, the mixtures were sonicated for 10 minutes. The particles had a diameter of 120 nm, PDI 0.233, and zeta potential of −52 mV.

In a second formulation, DHA (5.31 mg), 3.5 mg soy lecithin, 0.5 mg glycerol monostearate, and 1.75 mg vitamin E-TPGS (TPGS) were mixed and dispersed in 1 ml of de-ionized and filtered (0.22 μm) hot water (80° C.). The mixture was vortexed, sonicated for 10 minutes, and then maintained on an 80° C. hot plate while stirring at 800 rpm for 5 minutes. The emulsions were cooled to room temperature while stirring to form nanoparticles, which were further sonicated for 3 minutes. The particle size, polydispersity index (PDI), and zeta potential of the nanoparticles were determined using a Malvern Zeta Sizer Nano ZS (Westborough, Mass.). Results are summarized in Table 6. Morphology of the nanoparticles was examined using a transmission electron microscope (TEM) as shown in FIG. 13.

TABLE 6
Characterization of DHA-SLN.

| Particles diameter (nm) | PDI | Zeta potential (mV) |
|---|---|---|
| 128.6 ± 3.0 | 0.244 ± 0.010 | −52.0 ± 7.4 |

### DHA-Lecithin-GSM-TPGS-Tween 20

Docosahexaenoic acid (DHA, 5.4 mg), 3.5 mg soy lecithin, 0.5 mg glycerol monostearate, and 0.875 mg vitamin E-TPGS (TPGS) were mixed and dispersed in 800 μl of de-ionized and filtered (0.22 μm) hot water (80° C.). The mixture was vortexed, sonicated for 10 minutes, and then maintained on an 80° C. hot plate while stirring at 800 rpm for 5 minutes. Several concentrations of Tween 20 were separately dissolved (1 mg, 13.8 mg, 27.5 mg, and 55 mg) in 1 ml of hot water, and then 200 μl of these solutions were separately added dropwise into replicates of the DHA mixture to final concentrations of 0.1, 0.25, 0.5, and 1% (v/v) Tween 20. A sample without Tween 20 was prepared as described by adding 1 ml of de-ionized and filtered (0.22 μm) hot water (80° C.).

Particle diameter, polydispersity index (PDI), and zeta potential were determined using a Malvern Zeta Sizer Nano ZS (Westborough, Mass.). Results are summarized in Table 7. Increasing Tween 20 amount decreased the size of the resultant DHA-SLNs. However, DHA-SLNs prepared with low concentrations of Tween 20 (e.g. 0, 0.1, and 0.25%) were unstable and precipitated after 1 h. At 1% of Tween 20, the quality of the sample was not good enough for the Zeta Sizer Nano ZS to measure the zeta potential.

TABLE 7
Characterization of DHA-SLN with varying
concentrations of Tween 20.

| Tween 20 | Particle diameter | | Zeta potential |
|---|---|---|---|
| (%) | (nm) | PDI | (mV) |
| 0 | 140.8 ± 12.2 | 0.200 ± 0.04 | −48.2 ± 7.1 |
| 0.1 | 132.3 ± 3.6 | 0.227 ± 0.05 | −54.8 ± 3.7 |
| 0.25 | 121.8 ± 1.8 | 0.205 ± 0.01 | −50.2 ± 5.1 |
| 0.5 | 95.7 ± 1.6 | 0.236 ± 0.01 | −43.9 ± 4.1 |
| 1 | 47.6 ± 1.6 | 0.268 ± 0.01 | |

### DHA-Lecithin-TPGS

5.2 mg DHA, 3.5 mg soy lecithin, and 1.75 mg vitamin E-TPGS (TPGS) were mixed in 800 μl of de-ionized and filtered (0.22 μm) hot water (80° C.). The mixture was vortexed, sonicated for 5 minutes, and then maintained on a 80° C. hot plate while stirring at 800 rpm for 5 minutes. The emulsions were cooled to room temperature while stirring to form nanoparticles. Finally, the mixtures were sonicated for 3 minutes. The particles had a diameter of 122.7 nm, PDI 0.233, and zeta potential of −52.7 mV.

### DHA and TPGS Alone

5 mg DHA and 20 mg vitamin E-TPGS (TPGS) were mixed in 1000 μl of de-ionized and filtered (0.22 μm) hot water (80° C.). The mixture was vortexed, sonicated for 5 minutes, and then maintained on a 80° C. hot plate while stirring at 1000 rpm for 5 minutes. The mixtures were sonicated 2 minutes and cooled to room temperature while stirring to form nanoparticles. After 20 minutes, nanoparticles were filtered with PVDF 0.22 μm. The particles had a diameter of 52.4 nm and PDI 0.229.

### Example 4. SLN Formulations Having Alternative Active Compounds

### Docetaxel

Docetaxel (2.5 mg), 3.5 mg soy lecithin, 0.5 mg glycerol monostearate, and 0.875 mg vitamin E-TPGS (TPGS) were mixed and dispersed in 800 μl of de-ionized and filtered (0.22 μm) hot water (80° C.). The mixture was vortexed, sonicated for 10 minutes, and then maintained on an 80° C. hot plate while stirring at 800 rpm for 5 minutes. 55 mg Tween 20 was dissolved in 1 ml of hot water, and then 200 ml of this solution was added dropwise into the docetaxel mixture for a final concentration of 1% (v/v) Tween 20. The emulsions were cooled to room temperature while stirring to form nanoparticles, which were further sonicated for 30 to 45 minutes.

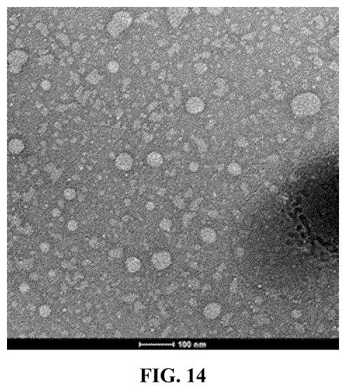

Particle diameter, polydispersity index (PDI), and zeta potential were determined using a Malvern Zeta Sizer Nano ZS (Westborough, Mass.). Results are summarized in Table 8. Morphology of docetaxel-SLNs were examined using a transmission electron microscope (TEM) as reported in FIG. 14. The nanoparticles prepared with 2.5 mg docetaxel have a good particle size (diameter) and polydispersity index (Table 8). TEM images of the docetaxel-SLNs showed that these particles were spherical (FIG. 14) with a particle diameter smaller than that determined by dynamic light scattering (Table 8).

TABLE 8
Characterization of docetaxel-SLNs.

| Particle diameter (nm) | PDI | Zeta potential (mV) |
|---|---|---|
| 280.03 ± 16.07 | 0.131 ± 0.02 | −36.90 ± 3.50 |

### DHA-Retinoic Acid

5 mg DHA, 20 mg vitamin E-TPGS (TPGS), and 250 μg retinoic acid on 1000 μl of de-ionized and filtered (0.22 μm) hot water (80° C.). The mixture was vortexed, sonicated for 3 minutes, and then maintained on a 80° C. hot plate while stirring at 1000 rpm for 5 minutes. The mixtures were sonicated 3 minutes and cooled to room temperature while stirring to form nanoparticles. After 20 minutes, nanoparticles were filtered with PVDF 0.22 μm. The particles had a diameter of 55.9 nm and PDI 0.233.

### Example 5. Comparative Formulations

### DHA-dFdC-PEG

DHA-dFdC (0, 4.56 or 3.5 mg), 3.5 mg soy lecithin, 0.5 mg glycerol monostearate, and 0.875 mg of 1,2-distearoyl-sn-glycero-3-phosphoethanolamine-N-[amino(polyethylene glycol)-2000] (DSPE-PEG2000) were mixed and dispersed in 800 μl of de-ionized and filtered (0.22 μm) hot water (80° C.). The mixture was vortexed, sonicated for 10 minutes, and then maintained on an 80° C. hot plate while stirring at 800 rpm for 5 minutes. Separately, 55 mg Tween 20 was dissolved in 1 ml of hot water, and then 200 ml of this solution was added dropwise into the mixture to reach a final concentration of 1% (v/v) Tween 20. The emulsions were cooled to room temperature while stirring to form nanoparticles, which were further sonicated for 30 to 45 minutes. The particle diameter and polydispersity index (PDI) of the nanoparticles were determined using a Malvern Zeta Sizer Nano ZS (Westborough, Mass.). Results are summarized in Table 9. When more than 4 mg of DHA-dFdC were used, undesirable results were obtained, such as the particle size was larger than 200 nm, and the polydispersity index was above 0.4.

TABLE 9
Characterization of DHA-dFdC-SLN containing DSPE-PEG2000

| Amount of DHA-dFdC (mg) | Particle diameter (nm) | PDI |
|---|---|---|
| 4.56 | 268.6 | 0.413 |
| 3.55 | 188.8 | 0.293 |
| 0 | 196.5 | 0.345 |

### DHA-dFdC-Vitamin E

Vitamin E has antioxidative activity. Feasibility of including vitamin E in the DHA-dFdC-solid lipid nanoparticles was examined DHA-dFdC (4.83 or 5 mg), 3.5 mg soy lecithin, 0.5 mg glycerol monostearate, 0.1 mg vitamin E, and 0.875 mg of DSPE-PEG2000 were mixed and dispersed in 800 μl of de-ionized and filtered (0.22 μm) hot water (80° C.). The mixture was vortexed, sonicated for 10 minutes, and then maintained on an 80° C. hot plate while stirring at 800 rpm for 5 minutes. Separately, 55 mg Tween 20 was dissolved in 1 ml of hot water, and then 200 ml of this solution was added dropwise into the mixture to reach a final concentration of 1% (v/v) Tween 20. The emulsions were cooled to room temperature while stirring to form nanoparticles, which were further sonicated for 30 to 45 minutes. The particle size, polydispersity index (PDI), and zeta potential of the nanoparticles were determined using a Malvern Zeta Sizer Nano ZS (Westborough, Mass.). Results are summarized in Table 10. The particle diameter was large and the polydispersity index was above 0.2.

TABLE 10
Characterization of DHA-dFdC-SLN containing vitamin E.

| Amount of | Particles | | Zeta |
|---|---|---|---|
| DHA-dFdC | diameter | | Potential |
| (mg) | (nm) | PDI | (mV) |
| 5 | 290.6 | 0.303 | −0.172 |
| 4.83 | 201.7 | 0.445 | −0.262 |

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

What is claimed is:

1. A nanoparticle composition comprising:
an active compound comprising a nucleobase analogue moiety covalently linked to an omega-3 polyunsaturated fatty acid moiety, or a pharmaceutically acceptable salt or prodrug thereof;
a pegylated vitamin E compound; and
at least one oil phase component.

2. The nanoparticle composition of claim 1, wherein the nucleobase analogue moiety comprises gemcitabine.

3. The nanoparticle composition of claim 1, wherein the omega-3 polyunsaturated fatty acid moiety comprises docosahexaenoic acid.

4. The nanoparticle composition of claim 1, wherein the active compound comprises a compound having a Formula I: 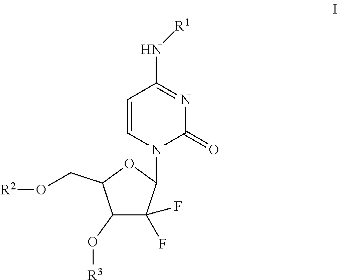
wherein R¹, R², and R³ are independently selected from hydrogen, halogen, hydroxyl, amino, thiol, thioalkyl, alkyl, alkenyl, alkynyl, haloalkyl, cycloalkyl, heterocycloalkyl, alkylaryl, aryl, alkylheteroaryl, heteroaryl, or omega-3 polyunsaturated fatty acid, any of which is optionally substituted with acetyl, alkyl, amino, amido, alkoxyl, alkylhydroxy, cycloalkyl, heterocycloalkyl, aryl, heteroaryl, carbonyl, halogen, hydroxyl, thiol, cyano, or nitro;
wherein at least one of R¹, R², or R³ comprises an omega-3 polyunsaturated fatty acid.

5. The nanoparticle composition of claim 1, wherein the active compound comprises 4-(N)-docosahexaenoyl 2′, 2′-difluorodeoxycytidine (DHA-dFdC).

6. The nanoparticle composition of claim 1, further comprising a solvent.

7. The nanoparticle composition of claim 1, wherein the nanoparticle composition comprises the active compound in an amount up to about 0.8 weight percent (w/v).

8. The nanoparticle composition of claim 1, wherein a nanoparticle of the nanoparticle composition comprises the active compound in an amount up to about 65 weight percent based on solids.

9. The nanoparticle composition of claim 1, wherein the pegylated vitamin E compound comprises a polyethylene glycol having a molecular weight ranging from about 200 g/mol to about 6000 g/mol, wherein the polyethylene glycol is esterified to a vitamin E succinate.

10. The nanoparticle composition of claim 1, wherein the pegylated vitamin E compound comprises D-α-tocopherol polyethylene glycol 1000 succinate (TPGS).

11. The nanoparticle composition of claim 1, wherein the oil phase component comprises lecithin.

12. The nanoparticle composition of claim 1, further comprising an additional oil phase component.

13. The nanoparticle composition of claim 12, wherein the additional oil phase component comprises a glycerol monostearate.

14. The nanoparticle composition of claim 1, further comprising an additional emulsifier.

15. The nanoparticle composition of claim 14, wherein the additional emulsifier comprises a polysorbate.

16. The nanoparticle composition of claim 1, wherein the nanoparticle has an average diameter of 200 nm or less.

17. A method of treating a subject with a disease comprising administering to the subject a therapeutically effective amount of a nanoparticle composition comprising:
an active compound comprising a nucleobase analogue moiety covalently linked to an omega-3 polyunsaturated fatty acid moiety, or a pharmaceutically acceptable salt or prodrug thereof;
a pegylated vitamin E compound; and
at least one oil phase component.

18. The method of claim 17, wherein the composition is administered parenterally.

19. The method of claim 17, wherein the composition is administered orally.

20. The method of any claim 17, wherein the disease comprises a tumor.

21. The method of claim 20, wherein the method reduces a rate of tumor growth.

22. The method of claim 20, wherein the method increases the amount of fibrous connective tissue within a tumor microenvironment.

23. A method of delivering an active compound to a biological cell comprising contacting the biological cell with a nanoparticle composition comprising:
the active compound comprising a nucleobase analogue moiety covalently linked to an omega-3 polyunsaturated fatty acid moiety, or a pharmaceutically acceptable salt or prodrug thereof;
a pegylated vitamin E compound; and
at least one oil phase component.

24. A method of making a nanoparticle comprising combining:
an active compound comprising a nucleobase analogue moiety covalently linked to an omega-3 polyunsaturated fatty acid moiety, or a pharmaceutically acceptable salt or prodrug thereof;
a pegylated vitamin E compound; and
at least one oil phase component.

25. The method of claim 24, wherein no organic solvents are used in the method.
